(19) United States
(12) Patent Application Publication
(10) Pub. No.: US 2026/0363735 A1
(43) Pub. Date: Nov. 19, 2026

(54) JOINT AUGMENTATION TECHNIQUE

(71) Applicant: Stryker Corporation, Kalamazoo, MI (US)

(72) Inventors: Markus Scheibel, Berlin (DE); Kyle Pilgeram, San Jose, CA (US)

(73) Assignee: Stryker Corporation, Kalamazoo, MI (US)

(21) Appl. No.: 19/677,916

(22) Filed: May 14, 2026

Related U.S. Application Data
(60) Provisional application No. 63/805,756, filed on May 14, 2025.
(60) Provisional application No. 63/899,635, filed on Oct. 15, 2025.

(51) Int. Cl. A61B 17/68; A61B 17/56; A61B 17/80; A61B 17/84; A61F 2/40

(52) U.S. Cl. CPC A61B 17/683; A61B 17/8061; A61B 17/842; A61F 2/40; A61B 2017/564

(57) ABSTRACT

A method for stabilizing a joint having a first bone and a second bone. The method comprising modifying a tissue of the joint to separate the tissue into a first portion and a second portion, the first portion of the modified tissue having a first end anchored to the second bone of the joint and a second end that is freely movable. The method further comprising preparing a tunnel in the first bone of the joint, guiding the second end of the first portion of the modified tissue through the tunnel, and guiding the second end of the first portion of the modified tissue to an anchor point at the second bone of the joint. The method further comprising anchoring the second end of the first portion of the modified tissue to the second bone at the anchor point.

### CROSS-REFERENCE TO RELATED PATENT APPLICATIONS

This application claims the benefit of and priority to U.S. Provisional Application No. 63/805,756, filed on May 14, 2025, and U.S. Provisional Application No. 63/899,635, filed on Oct. 15, 2025, the entire disclosures of which are hereby incorporated by reference herein.

### BACKGROUND

The present disclosure relates generally to systems and methods for orthopedic surgeries, for example that facilitate joint repair and/or stabilization. For example, the present disclosure may relate to systems and methods for acromioclavicular joint repair and stabilization.

Joint injuries are common among individuals. For example, a common injury of the shoulder girdle may be acromioclavicular joint (AC joint) dislocation also known as shoulder separation. Various open and minimally invasive surgical techniques have been developed to address injuries requiring surgical interventions, for example arthroscopically assisted techniques. While these techniques can be used to address AC joint injuries, loss of reduction surrounding these techniques still exist (e.g., implant irritation, joint instability, bone movement or translation, lack of graft integration or healing, etc.). Further, these techniques may utilize implantation and/or surgical techniques (e.g., implanting an augmentation and/or prosthetic device, joint or bone modification, including resection, etc.), which may lead to concerns surrounding joint and/or bone structure, stability, and/or healing. Therefore, it would be advantageous to have systems and methods that address these concerns, for example to provide improved clinical outcomes, more repeatable and/or successful repairs, reduced costs, and/or decreased time in surgery.

### SUMMARY

At least one embodiment relates to a method for stabilizing a joint having a first bone and a second bone. The method comprises modifying a tissue of the joint to separate the tissue into a first portion and a second portion, where the first portion of the modified tissue has a first end anchored to the second bone of the joint and a second end that is freely movable. The method comprises preparing a tunnel in the first bone of the joint, and guiding the second end of the first portion of the modified tissue through the tunnel. The method also comprises guiding the second end of the first portion of the modified tissue to an anchor point at the second bone of the joint, and anchoring the second end of the first portion of the modified tissue to the second bone at the anchor point.

At least one embodiment relates to a method for stabilizing a joint having a first bone, a second bone, and an augment material having a first end anchored to the second bone. The method comprises preparing the joint by modifying the augment material to allow a second end of the augment material, opposite the first end, to be freely movable. The method comprises preparing a tunnel in the first bone of the joint, and guiding the second end of the augment material through the tunnel. The method further comprises guiding the second end of the augment material to an anchor point at the second bone of the joint, and anchoring the second end of the augment material to the second bone at the anchor point.

At least one embodiment relates to a method for stabilizing a joint having a first bone and a second bone. The method comprises severing a tissue of the joint to separate the tissue into a first portion and a second portion, where the first portion has a first end anchored to the second bone of the joint and a second end that is freely movable. The method comprises performing a tensioning operation by applying a force to an elongated tensionable structure coupled to the first bone and the second bone to draw the first bone and the second bone toward one another, and preparing a tunnel in the first bone of the joint. The method also comprises guiding the second end of the first portion of the tissue through the tunnel, and anchoring the first portion of the tissue to the first bone via an anchor secured to the first portion of the tissue and received within the tunnel. The method further comprises guiding the second end of the first portion of the tissue to an anchor point at the second bone of the joint, and anchoring the second end of the first portion of the tissue to the second bone at the anchor point.

This summary is illustrative only and should not be regarded as limiting.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is an illustrative diagram of a joint of an individual, according to an embodiment.

FIG. 2 is a representation of an image of a thoracic region of an individual, according to an embodiment.

FIG. 3A is an illustrative diagram of a stage of a process for stabilizing a joint, according to an embodiment.

FIG. 3B is an illustrative diagram of a stage of a process for stabilizing a joint, according to an embodiment.

FIG. 3C is an illustrative diagram of a stage of a process for stabilizing a joint, according to an embodiment.

FIG. 4A is an illustrative diagram of another stage of a process for stabilizing a joint, according to an embodiment.

FIG. 4B is an illustrative diagram of another stage of a process for stabilizing a joint, according to an embodiment.

FIG. 5A is an illustrative diagram of another stage of a process for stabilizing a joint, according to an embodiment.

FIG. 5B is an illustrative diagram of another stage of a process for stabilizing a joint, according to an embodiment.

FIG. 6A is an illustrative diagram of another stage of a process for stabilizing a joint, according to an embodiment.

FIG. 6B is an illustrative diagram of another stage of a process for stabilizing a joint, according to an embodiment.

FIG. 6C is an illustrative diagram of another stage of a process for stabilizing a joint, according to an embodiment.

FIG. 6D is an illustrative diagram of another stage of a process for stabilizing a joint, according to an embodiment.

FIG. 7A is an illustrative diagram of another stage of a process for stabilizing a joint, according to an embodiment.

FIG. 7B is an illustrative diagram of another stage of a process for stabilizing a joint, according to an embodiment.

FIG. 8A is an illustrative diagram of another stage of a process for stabilizing a joint, according to an embodiment.

FIG. 8B is an illustrative diagram of another stage of a process for stabilizing a joint, according to an embodiment.

FIG. 8C is an illustrative diagram of another stage of a process for stabilizing a joint, according to an embodiment.

FIG. 9 is an illustrative diagram of another stage of a process for stabilizing a joint, according to an embodiment.

FIG. 10A is an illustrative diagram of another stage of a process for stabilizing a joint, according to an embodiment.

FIG. 10B is an illustrative diagram of another stage of a process for stabilizing a joint, according to an embodiment.

FIG. 11 is an illustrative diagram of another stage of a process for stabilizing a joint, according to an embodiment.

FIG. 12A is an illustrative diagram of another stage of a process for stabilizing a joint, according to an embodiment.

FIG. 12B is an illustrative diagram of another stage of a process for stabilizing a joint, according to an embodiment.

FIG. 12C is an illustrative diagram of another stage of a process for stabilizing a joint, according to an embodiment.

FIG. 12D is an illustrative diagram of another stage of a process for stabilizing a joint, according to an embodiment.

FIG. 12E is an illustrative diagram of another stage of a process for stabilizing a joint, according to an embodiment.

FIG. 12F is an illustrative diagram of another stage of a process for stabilizing a joint, according to an embodiment.

FIG. 12G is an illustrative diagram of another stage of a process for stabilizing a joint, according to an embodiment.

FIG. 12H is an illustrative diagram of another stage of a process for stabilizing a joint, according to an embodiment.

FIG. 12I is an illustrative diagram of another stage of a process for stabilizing a joint, according to an embodiment.

FIG. 13A is an illustrative diagram of a device used for stabilizing a joint, according to an embodiment.

FIG. 13B is an illustrative diagram of another device used for stabilizing a joint, according to an embodiment.

FIG. 14 is an illustrative diagram of another device used for stabilizing a joint, according to an embodiment.

FIG. 15A is an illustrative diagram of another device used for stabilizing a joint, according to an embodiment.

FIG. 15B is an illustrative diagram of another device used for stabilizing a joint, according to an embodiment.

FIG. 15C is an illustrative diagram of another device used for stabilizing a joint, according to an embodiment.

FIG. 15D is an illustrative diagram of another device used for stabilizing a joint, according to an embodiment.

FIG. 15E is an illustrative diagram of another device used for stabilizing a joint, according to an embodiment.

FIG. 16 is a flow chart of a method for stabilizing a joint, according to an embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the Figures, the systems and methods described herein provide a joint repair and/or stabilization technique, which utilizes a graft secured between a first bone and a second bone. The graft can be allograft, xenograft and/or autograft. The graft may be artificial, natural, and/or a combination (e.g., biocomposite, etc.). In one example, the tissue may be an autograft (e.g., a long head of the biceps tendon, etc.) that is routed from an anchor (e.g., an anchor point of the tendon, an anchor point at the scapula, a proximal anchor point at a tubercle of the glenoid cavity, etc.), to and/or through a first bone (e.g., the clavicle, etc.), and attached at a second bone (e.g., a point at the scapula, an acromion of the scapula, etc.). For instance, as provided in the example illustrated herein, the long head of the biceps tendon may already be connected to the scapula, though alternatively, the tendon might need to be reattached to the scapula if the soft tissue was damaged or otherwise purposefully detached prior to its use as a graft.

Advantageously, the systems and methods described herein provide a technique that utilizes autograft tissue to augment anatomical joint features (e.g., joint bones, muscles, ligaments, etc.), which offers increased healing potential, lower cost, and/or reduced disease transmission potential. Further, the systems and methods described herein provide a technique that utilizes existing anatomical attachment points (e.g., an anchor point, an anchor point of the autograft tissue, etc.), which may be leveraged to decrease the need for healing at such an attachment point while also providing increased stability to the joint and/or further offering increased healing potential, for example compared to traditional surgical techniques.

As described herein, the term “elongated tensionable structure,” “elongated structure,” “tensionable structure,” “elongate structure,” or similar terms, may generally refer to an elongate structure or component that is designed to carry, support, resist, and/or hold a load, for example by being put under tension or being tensioned. For example, such an elongated tensionable structure (or similar terms) may be a suture, a tape, such as suture tape, an augment, a tissue, a wire, a cable made of a plurality of wires, and/or any combination of the aforementioned options. As described herein, the elongated tensionable structure may be a braided suture, for example formed of ultra-high molecular weight polyethylene (UHMWPE), polyester (PET), and/or other materials. The elongated tensionable structure may be formed entirely of or in part of biodegradable polymers, and/or may include biologic components (e.g., a biocomposite suture material). The elongated tensionable structure may be formed of other potential materials and/or combinations of materials, for example synthetic material, synthetic material designed to stretch (e.g., a FlexBand® material, etc.), a synthetic-augment hybrid material, a synthetic-allograft material hybrid, a biocomposite material, coated material, etc. For example, in some embodiments the materials and/or combinations of materials may include a synthetic-augment hybrid material designed to stretch, a biocomposite material designed to stretch, a coated material designed to stretch, etc. All such variations and/or combinations are contemplated herein.

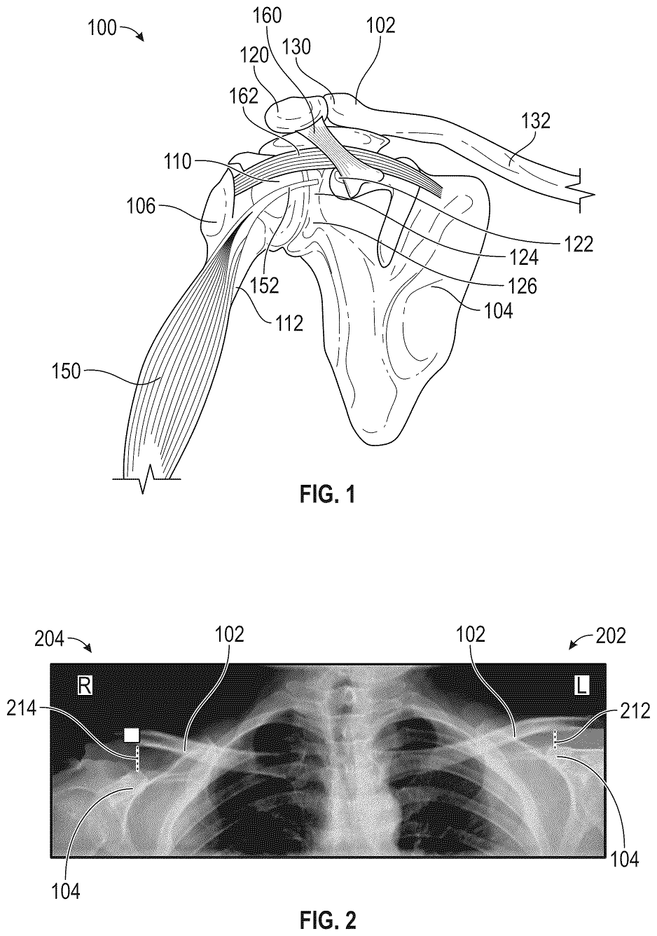

Referring now to FIG. 1, an anatomical region, illustrated as a shoulder 100, is shown, according to an exemplary embodiment. In an exemplary embodiment, the shoulder 100 includes one or more joints, for example an AC joint and a glenohumeral joint. It should be understood that while the shoulder 100 is shown and described herein as having certain anatomical features (e.g., joints, bones, tissues, etc.), it is contemplated that in other embodiments the shoulder 100 is another suitable anatomical region, joint, and/or feature (e.g., hip, knee, ankle, wrist, spinal, etc. joint or anatomical feature, etc.).

The shoulder 100 is shown to include a plurality of anatomical features, structures, and/or components. For example, the shoulder 100 is shown to include a first bone, shown as a clavicle 102, a second bone, shown as a scapula 104, and a third bone, shown as a humerus 106. In an exemplary embodiment, the shoulder 100 (e.g., the bones of the shoulder 100, etc.) includes one or more anatomical features, which, for example, facilitate connection, stabilization, and/or formation of the shoulder 100. For example, the shoulder 100 may include one or more protrusions, projections, tubercles, cavities, fossas, borders, surfaces, and/or any other suitable anatomical feature or structure.

As shown, the humerus 106 includes one or more bone structures, and/or other anatomical features. For example, the humerus 106 is shown to include a first end or upper end, shown as humerus head 110, and extends in a distal direction to a second end or lower end which forms part of the elbow (not shown). As described herein, the humerus head 110 is configured to articulate (e.g., engage, interface, etc.) with the scapula 104 (e.g., a cavity of the scapula 104, etc.), for example as part of the shoulder 100 (e.g., the glenohumeral joint, etc.). The humerus 106 is also shown to include a body or shaft, shown as humerus body 112. The humerus body 112 is shown to extend from the humerus head 110, for example distally toward a second end or lower end.

The scapula 104 is also shown to include one or more bones, bone structures, and/or other anatomical features. For example, the scapula 104 is shown to include a first projection, shown as an acromion 120, and a second projection, shown as a coracoid process 122. The acromion 120 is shown to extend from a posterior portion of the scapula 104, and the coracoid process 122 is shown to extend from an anterior portion of the scapula 104. As described herein, the acromion 120 is configured to articulate (e.g., engage, interface, etc.) with the clavicle 102 (e.g., an end of the clavicle, etc.), for example as part of the shoulder 100 (e.g., the AC joint, etc.). The scapula 104 may further include a cavity or fossa (e.g., a glenoid cavity, etc.). The glenoid cavity may have and/or be defined by one or more surfaces, edges, ridges, etc. For example, the glenoid cavity may be adjacent to and/or define a projection or protrusion, shown as a tubercle 124, and/or may be defined by an edge or ridge, shown as a glenoid rim 126 (e.g., an anterior and/or medial portion of the glenoid, etc.). Further, the glenoid cavity may be configured to articulate (e.g., engage, interface, receive, etc.) with the humerus 106 (e.g., the humerus head 110, etc.), for example as part of the shoulder 100 (e.g., the glenohumeral joint, etc.).

The clavicle 102 is also shown to include one or more bones, bone structures, and/or other anatomical features. For example, the clavicle 102 is shown to include a first end, shown as acromial end 130, and a body or shaft, shown as clavicle body 132. As described herein, the acromial end 130 is configured to articulate (e.g., engage, interface, tec.) with the scapula 104 (e.g., the acromion 120, etc.), for example as part of the shoulder 100 (e.g., the AC joint, etc.). Further, the clavicle body 132 is shown to extend from the acromial end 130, for example medially toward a second end or medial end (e.g., sternal end, etc.).

According to an exemplary embodiment, one or more features of the shoulder 100 may be connected via one or more anatomical features, structures, and/or components (e.g., tissues, tendons, ligaments, etc.). For example, the shoulder 100 is shown to include a first tissue or tendon, shown as a biceps 150. The biceps 150 may be a long head of a biceps muscle. The biceps 150 is shown to include an end or proximal end, shown as a first end 152, and may extend from the first end toward a second or distal end. The first end 152 may be or include tissue, for example transitioning to the biceps tendon. As described herein, the biceps 150 (e.g., the first end 152) may connect (e.g., originate, anchor, attach, etc.) to the scapula 104, for example at the tubercle 124 (e.g., a tubercle of the glenoid cavity, a supraglenoid tubercle, etc.).

As shown, the shoulder 100 may include one or more additional anatomical features, structures, and/or components. For example, the shoulder 100 is shown to include a second tissue, shown as a coracoacromial ligament 160. The coracoacromial ligament 160 is shown to connect the coracoid process 122 and the acromion 120. Further, the shoulder 100 is shown to include a third tissue or muscle, shown as tissue 162. The tissue 162 may be a supraspinatus muscle, or another suitable muscle or ligament, for example to connect the scapula 104 and the humerus 106. It should be understood that while the shoulder 100 is described herein as having certain anatomical features, it is contemplated that the shoulder 100 (e.g., an AC joint, a glenohumeral joint, etc.) has any other suitable anatomical features, as would be understood by a person skilled in the art.

Referring now to FIG. 2, a representation of an image (e.g., an X-ray image, etc.) of a thoracic region of an individual is shown, according to an exemplary embodiment. As shown, the individual includes a first shoulder, shown as a left shoulder 202, and a second shoulder, shown as a right shoulder 204. The shoulders 202, 204 may be similar to or the same as the shoulder 100 of FIG. 1 (e.g., including AC joints, glenohumeral joints, etc.).

According to an exemplary embodiment, the shoulder 202 exhibits a first distance, shown as distance 212, between the clavicle 102 and the scapula 104 (e.g., the coracoid process 122, a first coracoclavicular distance, etc.). Further, the shoulder 204 exhibits a second distance, shown as distance 214, between the clavicle 102 and the scapula 104 (e.g., the coracoid process 122, a second coracoclavicular distance, etc.). In some embodiments, the shoulder 204 illustrates a shoulder with trauma or injury (e.g., dislocation, stress, strain, vertical loss of reduction, lateral loss of reduction, etc.). For example, the shoulder 204 is shown to exhibit a distance 214 that is greater than the distance 212 of the shoulder 202 (e.g., a vertical loss of reduction, etc.). Further, the shoulder 204 may exhibit instability and/or directional movement (e.g., bidirectional movement, posterior/anterior movement, etc.) compared to the shoulder 202. According to an exemplary embodiment, the systems and methods described herein may be implemented to address loss of reduction associated with a shoulder and/or associated features (e.g., the shoulder 204, a dislocated AC joint, separated shoulder, etc.).

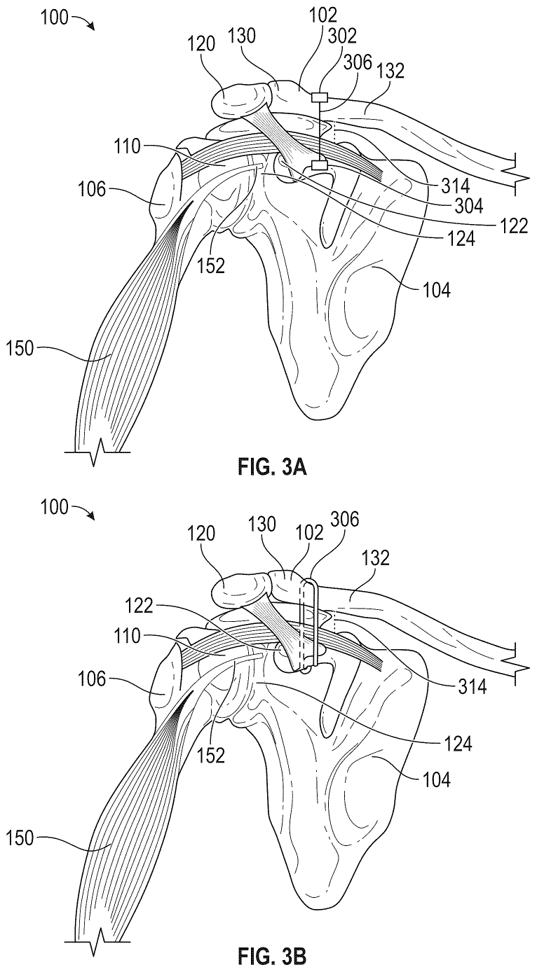

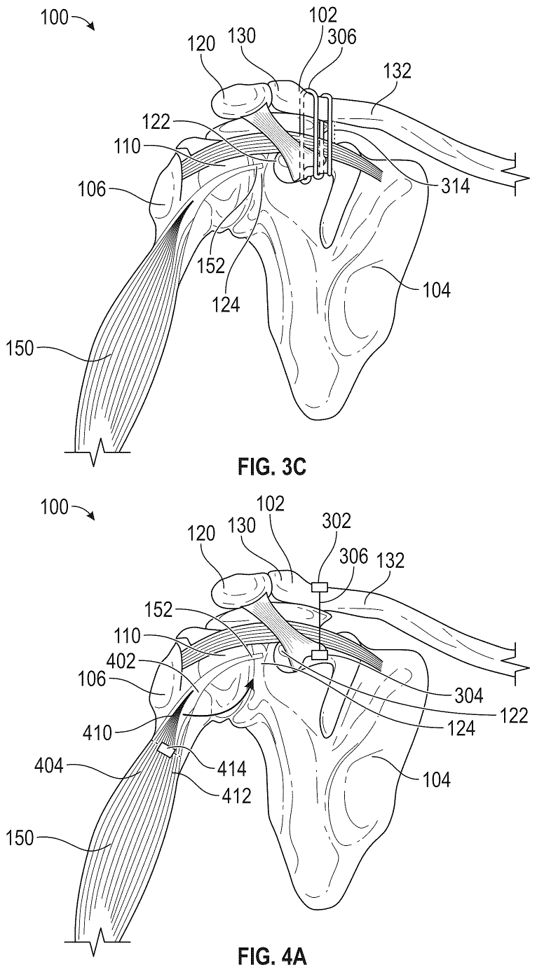

Referring generally to FIGS. 3A-16, various aspects of systems and methods for repairing and/or stabilizing one or more features of a shoulder (e.g., a joint, etc.) are shown. Specifically, FIGS. 3A-16 illustrate exemplary diagrams of various stages throughout processes for repairing and/or stabilizing features of the shoulder 100 of FIG. 1 (e.g., an AC joint, a glenohumeral joint, etc.). The exemplary depictions of FIGS. 3A-16 may be implemented in various combinations as desired by an operator, some of which are described herein, using one or more components, processes, and/or techniques, for example associated with one or more diagnostic and/or surgical techniques.

For example, the aspects illustrated in FIGS. 3A-16 may include performing one or more diagnostic techniques. In some embodiments, a diagnostic arthroscopy is performed on a joint (e.g., the shoulder 100, etc.), for example via a posterior portal to assess joint conditions (e.g., pathogens, concomitant glenohumeral pathologies, inflammation, etc.). Further, in some embodiments one or more surgical techniques may be performed. For example, and as will be described herein, a tenotomy or tenodesis may be performed. According to various embodiments, a Bankart repair and/or a rotator cuff repair could also be performed.

It should be understood that the diagnostic and/or surgical techniques described herein may be implemented using one or more surgical devices, components, and/or features, for example, cameras, resection devices (e.g., drills, burs, ablation devices, etc.), grasping devices, elongated tensionable structures, anchors, guidewires, stabilization wires, and/or any other suitable surgical component or device. In this regard, it is contemplated that any and/or all of the processes illustrated in FIGS. 3A-16 may be implemented using, or in combination with, any and/or all of the devices described herein. Additionally, the processes illustrated in FIGS. 3A-16 and discussed herein may be performed arthroscopically or as part of an open procedure.

Referring now to FIGS. 3A-3C, the process may include performing a tensioning technique, according to an exemplary embodiment. In exemplary embodiments, the tensioning technique is implemented to reduce the clavicle 102, for example to reduce (e.g., decrease, etc.) a distance (e.g., vertical distance, lateral distance, etc.) between the clavicle 102 and the scapula 104 and/or to provide proper alignment between the clavicle 102 and the scapula 104. For example, the tensioning technique illustrated in FIGS. 3A-3C may be implemented to reduce (e.g., decrease, etc.) a distance between the clavicle 102 and the coracoid process 122 (e.g., a coracoclavicular distance, etc., such as distance 314). According to an exemplary embodiment, and as will be described herein, the exemplary tensioning technique illustrated in FIGS. 3A-3C may be implemented to reduce the clavicle 102, for example to facilitate the techniques (e.g., a routed biceps tendon technique, a joint augmentation technique, a tissue augmentation technique, etc.) described elsewhere herein. In some embodiments, the systems and methods described herein may use additional and/or different techniques (e.g., tensioning techniques, etc.) relative to those illustrated in FIGS. 3A-3C, though generally such a tensioning technique is optional relative to the systems and methods described herein.

In various embodiments, and as shown in FIG. 3A, a first anchor, shown as anchor 302, may be coupled with the clavicle 102, and a second anchor, shown as anchor 304, may be coupled with the scapula 104. The first anchor 302 is shown to be implanted (e.g., inserted, etc.) into the clavicle body 132 (e.g., a superior portion of the clavicle body 132, etc.), and the second anchor 304 is shown to be implanted (e.g., inserted, etc.) into the coracoid process 122 (e.g., a base of the coracoid process 122, an inferior portion of the coracoid process 122, etc.). In some embodiments, the first anchor 302 and/or the second anchor 304 are inserted into a hole (e.g., bore, channel, cavity, etc.) in bone (e.g., the clavicle 102, the scapula 104, etc.), for example created via a drill or another suitable device described herein (e.g., burr, etc.). For example, the first anchor 302 may be inserted into a first hole (e.g., in the clavicle 102, etc.) and the second anchor 304 may be inserted into a second hole (e.g., in the scapula 104, etc.). In various embodiments, one or more of the anchors (e.g., the first anchor 302, the second anchor 304, etc.) is/are pins; however, in other embodiments one or more of the anchors is/are a top hat button, a drop crotch button, and/or another suitable anchor described herein. In other embodiments, the first anchor 302 and/or the second anchor 304 are inserted using other suitable techniques.

As shown, the first anchor 302 and the second anchor 304 can be connected, for example via an elongated tensionable structure, shown as elongated tensionable structure 306. In some embodiments, the elongated tensionable structure 306 is configured to be manipulated (e.g., tensioned, pulled, drawn, etc.). For example, a force may be applied to the elongated tensionable structure 306 (e.g., 50N, 75N, 80 N, 100 N, etc.), thereby reducing a distance between the first anchor 302 and the second anchor 304. In this regard, a force may be applied to the elongated tensionable structure 306 to reduce a distance (e.g., shown as distance 314) between the clavicle 102 and the scapula 104 (e.g., the coracoid process 122, a coracoclavicular distance, etc.). In some embodiments, the elongated tensionable structure 306 is manipulated by applying a force to an end of the elongated tensionable structure 306 at the clavicle 102 (e.g., pulling a superior end of the elongated tensionable structure 306, pulling a superior end of the elongated tensionable structure 306 at the clavicle 102, which may or may not be assisted with the use of a tensioning instrument, etc.). In other embodiments, the elongated tensionable structure 306 is otherwise manipulated (e.g., drawn, sinched, etc.) to draw the first anchor 302 and/or the second anchor 304 toward one another, thereby reducing the distance between the clavicle 102 and the scapula 104 (e.g., the coracoid process 122, a coracoclavicular distance, etc.).

In various embodiments disclosed herein, the anchors (e.g., the first anchor 302, the second anchor 304, etc.) may be hard (e.g., formed of PEEK or the like such as G-Force™ interference screws, metal, etc.) or soft (e.g., all-suture anchors such as ICONIX® (Stryker Corporation, Kalamazoo, MI), as shown and described in U.S. Pat. No. 9,445,803, the entirety of which is incorporated by reference herein). The anchors (e.g., the first anchor 302, the second anchor 304, etc.) may be a hard anchor, soft anchor, a button anchor (e.g., such as standard and slotted button anchors used in the ProCinch® system), a screw-in anchor, a threaded anchor, a wedge anchor, an interference anchor, and/or another suitable anchor, or any combination thereof. For example, the anchors (e.g., the first anchor 302, the second anchor 304, etc.) may be a top hat anchor (e.g., or other anchor that serves as a sheath to prevent wear caused by the elongated tensionable structure directly rubbing against the bone, etc.), a one-way trap door anchor (e.g., on button anchors, hard body anchors, etc.), a finger-trap anchor (e.g., formed by a suture-through-suture configuration or other splice configurations, which may also be attached to hard body anchors, all-suture anchors, button anchors, etc., e.g., ICONIX® and ICONIX® Knotless (Stryker Corp., Kalamazoo, MI)), expandable anchors, anchors with cleats or one-way suture fixation mechanisms (e.g., Piton™ anchors (Stryker Corp., Kalamazoo, MI)), and/or any other suitable anchor. Further, the anchors (e.g., the first anchor 302, the second anchor 304, etc.) may include an eyelet, cannulation, opening, aperture, guide, tube, arm (e.g., projection, protrusion, etc.), and/or another elongated tensionable structure, for example to allow one or more elongated tensionable structures (e.g., the elongated tensionable structure 306, etc.) to be connected thereto. Yet further, the anchors (e.g., the first anchor 302, the second anchor 304, etc.) may include one or more openings, guides, tubes, sheaths, arms, channels, grooves, and/or another suitable component or structure, for example to facilitate coupling and/or movement of one or more tissues (e.g., a tendon, a ligament, a tissue, etc.), elongated tensionable structures, and/or other prosthetic devices to and/or through the anchors.

In various embodiments disclosed herein, the elongated tensionable structure (e.g., the elongated tensionable structure 306, etc.) may be a braided suture, for example formed of ultra-high molecular weight polyethylene (UHMWPE), polyester (PET), and/or other materials. In other embodiments, the elongated tensionable structure (e.g., the elongated tensionable structure 306, etc.) is another suitable suture (e.g., a round suture, a tape suture, etc.) for connecting one or more anchors (e.g., the first anchor 302, the second anchor 304, etc.) and/or anatomical structures, which may be generally known in the art (e.g., the orthopedic surgery art, etc.). Other common elongated tensionable structure materials may include sutures formed entirely of or in part of biodegradable polymers, and/or may include biologic components (e.g., a biocomposite suture material). Still other potential materials and/or combinations of materials may be used in this tensioning technique, for example synthetic material, synthetic material designed to stretch (e.g., a FlexBand® material, etc.), a synthetic-augment hybrid material, a synthetic-allograft material hybrid, a biocomposite material, coated material, etc. For example, in some embodiments the materials and/or combinations of materials may include a synthetic-augment hybrid material designed to stretch, a biocomposite material designed to stretch, a coated material designed to stretch, etc.

In various embodiments disclosed herein, the anchors (e.g., the first anchor 302, the second anchor 304, etc.) and/or the elongated tensionable structure (e.g., the elongated tensionable structure 306, etc.) may be implemented alone and/or as part of a system (e.g., in combination, etc.). For example, the anchors and/or elongated tensionable structures may be part of a knotless anchor platform system, such as in the AlphaVent Knotless® system, the ReelX STT® system, the Knotilus+® system, the Iconix Knotless® system. Still other potential combinations of components and/or materials may be used in the one or more techniques described herein.

As described herein, in some embodiments the tensioning technique includes additional and/or different components relative to those illustrated in FIGS. 3A-3C. For example, in some implementations a ProCinch® suture may be used to perform the tensioning technique. In some embodiments, the tensioning technique includes coupling a plurality of anchors to a bone. For example, in some embodiments, the tensioning technique may include coupling two or more anchors to the clavicle 102, coupling two or more anchors to the scapula 104, and coupling the anchors via at least one elongated tensionable structure (e.g., the elongated tensionable structure 306, etc.). In other embodiments, the tensioning technique may include coupling a plurality of anchors (e.g., the first anchor 302, another anchor, etc.) to the clavicle 102, an anchor to the scapula 104 (e.g., the second anchor 304 to the coracoid process 122, etc.), and coupling the anchors via at least one elongated tensionable structure (e.g., the elongated tensionable structure 306, etc.). Similarly, the tensioning technique may include coupling a plurality of anchors (e.g., the second anchor 304, another anchor, etc.) to the scapula 104, at least one anchor to the clavicle 102 (e.g., the first anchor 302), and coupling the anchors via at least one elongated tensionable structure (e.g., the elongated tensionable structure 306, etc.). In such configurations, the at least one elongated tensionable structure may form a configuration (e.g., Y-shaped, X-shaped, etc. configuration), for example while connecting the anchors.

In other embodiments, the tensioning technique includes coupling one anchor to a bone and an elongated tensionable structure associated with the anchor to another bone. For example, the tensioning technique may include coupling an anchor (e.g., the first anchor 302, etc.) to the clavicle 102, and coupling the elongated tensionable structure (e.g., the elongated tensionable structure 306, etc.) to another bone (e.g., extending the elongated tensionable structure 306 to, around, and/or through the scapula 104, the coracoid process 122, etc.). Similarly, the tensioning technique may include coupling an anchor (e.g., the second anchor 304, etc.) to the scapula 104 (e.g., the coracoid process 122, etc.), and coupling the elongated tensionable structure 306 (e.g., the elongated tensionable structure 306, etc.) to another bone (e.g., extending the elongated tensionable structure 306 to, around, and/or through the clavicle 102, etc.). In such configurations, the elongated tensionable structure (e.g., the elongated tensionable structure 306, etc.) may be coupled with the anchor and/or the other bone, for example to form an elongated tensionable structure configuration (e.g., an X-shaped, a wound, a crossed, a parallel, a looped, etc. configuration).

In yet other embodiments, and as shown in at least FIGS. 3B-3C, the tensioning technique includes coupling an elongated tensionable structure between two bones. In this sense, in some embodiments, the elongated tensionable structure 306 may be wrapped around the clavicle 102 and/or the scapula 104 (e.g., the coracoid process 122, etc.), for example to reduce an acromioclavicular separation or the distance 314. Such a configuration may establish a cerclage around the two bone portions. As shown in at least FIG. 3B, the elongated tensionable structure 306 is wrapped around the coracoid process 122 at least one time, and/or the elongated tensionable structure 306 is wrapped around the clavicle 102 at least one time. As shown in at least FIG. 3C, in some embodiments the elongated tensionable structure 306 is wrapped around the coracoid process 122 two or more times, and/or the elongated tensionable structure 306 is wrapped around the clavicle 102 two or more times. In various embodiments, once the elongated tensionable structure 306 is so positioned, it may be secured (e.g., to the coracoid process 122, the clavicle 102, itself, another anatomical feature, etc.), for example via one or more knots (e.g., to secure structure 306 to itself), one or more clasps (e.g., to secure structure 306 to itself), and/or one or more knotless techniques, as described elsewhere herein. One or more instruments may be used to position the elongated tensionable structure in this fashion, such as suture passers, suture graspers, shuttle mechanisms, and the like as known in the art.

In various embodiments, the tensioning technique may include coupling an elongated tensionable structure (e.g., the elongated tensionable structure 306, etc.) to the clavicle 102 and the scapula 104 (e.g., the coracoid process 122, etc.), for example by extending the elongated tensionable structure 306 to, around, and/or through the clavicle 102 and the scapula 104 (e.g., the coracoid process 122, etc.). In such configurations, the elongated tensionable structure (e.g., the elongated tensionable structure 306, etc.) may be coupled with the bones, for example to form an elongated tensionable structure configuration. As discussed herein, the elongated tensionable structure configuration may include the elongated tensionable structure 306 wrapped around the coracoid process 122 and the clavicle 102 one time (as shown in at least FIG. 3B), the elongated tensionable structure 306 wrapped around the coracoid process 122 and the clavicle 102 two or more times (as shown in at least FIG. 3C), and/or another suitable configuration of the elongated tensionable structure 306 (e.g., an X-shaped, a wound, a crossed, a parallel, a looped, etc. configuration). In some embodiments, the elongated tensionable structure (e.g., the elongated tensionable structure 306, etc.) may extend to and/or through one or more channels in a bone (e.g., a first channel in the clavicle 102, a second channel in the scapula 104, etc.). In such configurations, the hole may include a sheath and/or another protective material or component, for example to protect the elongated tensionable structure and/or adjacent bone from damage or trauma (e.g., cuts, tearing, etc.).

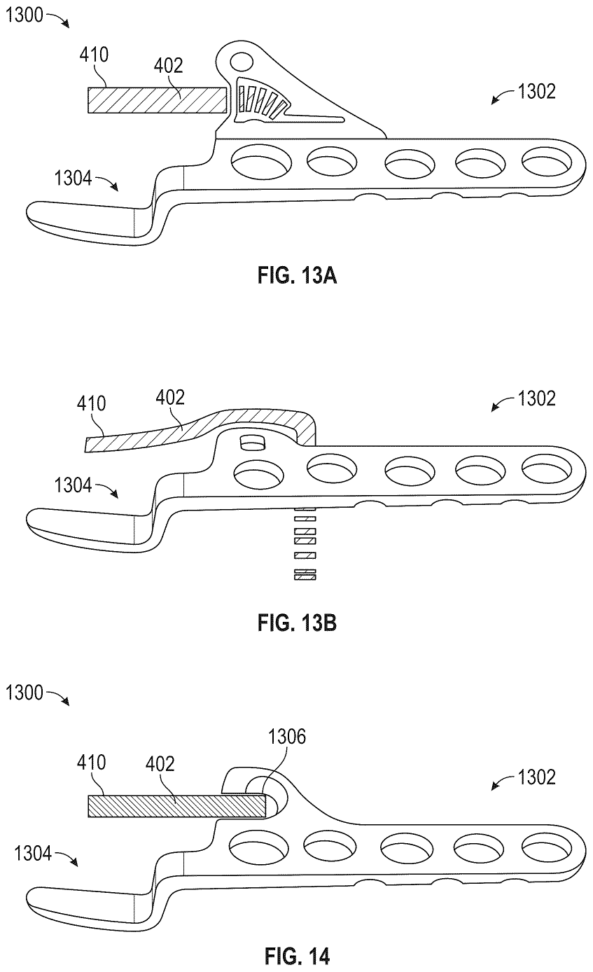

In other embodiments, the tensioning technique includes additional and/or different components. For example, the tensioning technique may include a prosthetic device coupled to a first bone (e.g., a plate coupled to the clavicle 102, a sheath, clip, or tube coupled to the clavicle 102, etc.). An elongated tensionable structure (e.g., the elongated tensionable structure 306, etc.) may be coupled to the prosthetic device, and may extend to, around, and/or through a second bone (e.g., the scapula 104, the coracoid process 122, etc.). A force may be applied to the elongated tensionable structure (e.g., the elongated tensionable structure 306, etc.), for example to decrease a distance between the first bone (e.g., the clavicle 102, etc.) and the second bone (e.g., the scapula 104, the coracoid process 122, etc.). In some embodiments, the prosthetic device includes one or more components (e.g., a guide, tube, channel, tunnel, arm, etc.), for example to protect a component received by the prosthetic device (e.g., an elongated tensionable structure, a tendon, a tissue, etc.) from damage (e.g., wear, tear, harm, etc.) associated with interfacing or engaging adjacent structures (e.g., bone surfaces or edges, edges or surfaces of tunnels in a bone, implanted and/or adjacent prosthetic devices, etc.). In some embodiments, the prosthetic device is a plate (e.g., an elongated plate, etc.), a clip, a hook, a hook plate (e.g., as shown with reference to FIGS. 13A-14, etc.), and/or another suitable device, described herein.

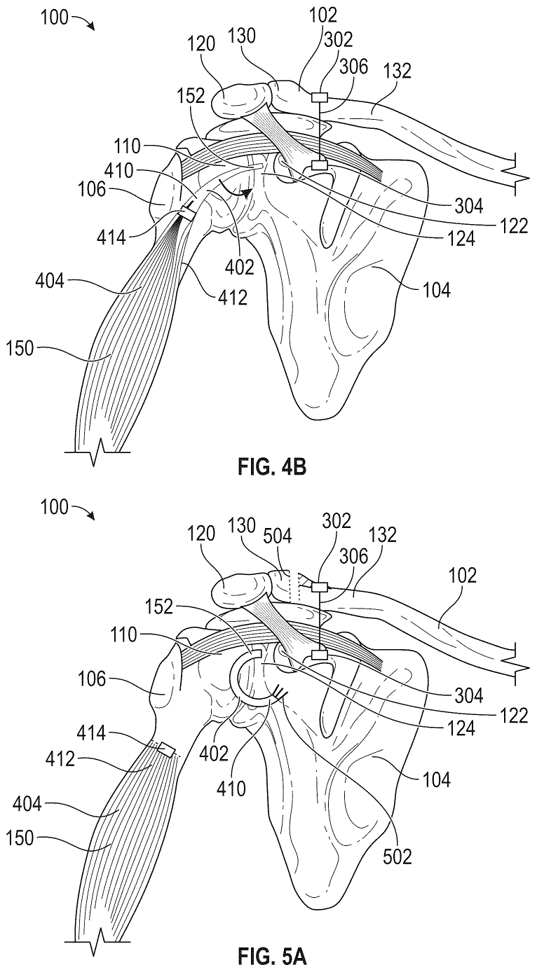

Referring now to FIGS. 4A-4B, the process may include modifying one or more tissues of the shoulder 100, according to exemplary embodiments. For example, a tenotomy or tenodesis may be performed on the biceps 150. In some embodiments, a tenotomy or tenodesis is performed on the biceps 150 to separate (e.g., cut, sever, divide, etc.) the biceps 150 into an upper or medial portion, shown as a first portion 402, and a lower or distal portion, shown as a second portion 404. In exemplary embodiments, the techniques illustrated by the illustrations of FIGS. 4A-4B are implemented using a suitable surgical technique (e.g., a subpectoral technique (such as in FIG. 4A), a suprapectoral technique (such as in FIG. 4B), etc.), for example to ensure a predetermined size (e.g., length, distance, etc.) of the first portion 402.

As shown in the technique illustrated by the illustration of FIG. 4A, a subpectoral tenotomy or tenodesis may be performed on the biceps 150. Further, as shown in the technique illustrated by the illustration of FIG. 4B, a suprapectoral tenotomy or tenodesis may be performed on the biceps 150. As discussed herein, a “subpectoral” tenotomy or tenodesis refers to a process of cutting or separating the biceps at a point below an attachment point of the pectoralis major at the humerus. Conversely, as discussed herein, a “suprapectoral” tenotomy or tenodesis refers to a process of cutting or separating the biceps at a point above the attachment point of the pectoralis major at the humerus.

In this sense, and as illustrated, the suprapectoral tenotomy or tenodesis technique includes modifying the biceps 150 closer to the proximal anchor of the long head of the biceps 150, thereby resulting in a shorter graft (e.g., a shorter first portion 402, etc.) compared to other techniques (e.g., the subpectoral technique illustrated in FIG. 4A). It should be understood that any and/or all of the techniques described herein may be implemented using any suitable surgical technique, including the subpectoral tenotomy or tenodesis illustrated in FIG. 4A or the suprapectoral tenotomy or tenodesis illustrated in FIG. 4B. It should also be understood that this aspect of the processes may be performed before or after the aforementioned tensioning techniques, or may be performed in lieu of the tensioning techniques (e.g., the tensioning techniques may not be performed at all).

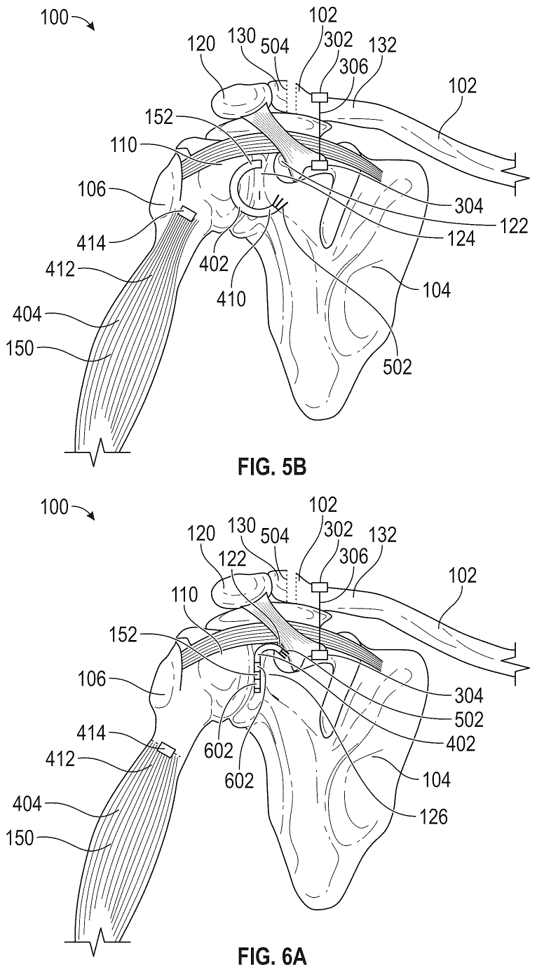

In both variations illustrated in FIGS. 4A-4B, the first portion 402 is shown to include a first end (e.g., the first end 152, etc.), which is connected (e.g., attached, anchored, etc.) to the scapula 104 (e.g., the tubercle 124 of the glenoid cavity, etc.). In some instances, the first end 152 is connected to the scapula 104 via a natural attachment (e.g., attached or connected without the assistance of additional anchoring devices, etc.), for example at its native site (e.g., the tubercle 124, etc.). In instances where the first end 152 is not attached to bone (e.g., the scapula 104, the tubercle 124 of the glenoid cavity, etc.), the first end 152 may be attached (e.g., reattached) to bone at its native site, or at another location as known in the art (e.g., via use of an interference screw or other anchor, use of an elongated tensionable structure, etc.). Following the tenotomy or tenodesis, the first portion 402 may further include a second end or free end, shown as second end 410 (e.g., in at least FIGS. 5A-5B), distal to and/or opposite the first end 152. The second portion 404 is shown to include a first end 412, which following the tenotomy may be a free end, and a second or distal end opposite the first end 412. In some embodiments, and as discussed herein, as part of and/or following a tenodesis, the first end 412 of the second portion 404 may be secured (e.g., attached, coupled, anchored, fixated, fixed, etc.) to a bone or bone structure (e.g., the humerus 106, etc.).

As described elsewhere herein, it should be understood that in other embodiments, the first portion 402 and/or the second portion 404 (e.g., ends of the first portion 402, ends of the second portion 404, etc.) may be connected to one or more bones at a native location, and/or another suitable location. For example, in instances where an allograft and/or autograft is implemented, either ends of any suitable tissue or graft (e.g., the first end 152 of the first portion 402, the second end 410 of the first portion 402, etc.) may be connected (e.g., secured, anchored, etc.) to a bone (e.g., the scapula 104, the tubercle 124 of the scapula 104, the glenoid rim 126 of the scapula 104, etc.).

In addition, it should be understood that as described herein, the first end 152 of the first portion 402 may be or describe one or more portions or regions (e.g., an end, a middle, an edge, etc.) of the first portion 402 that is secured (e.g., attached, fixed, coupled, looped, anchored, etc.) relative to a bone or bone structure. Further, it should be understood that the second end 410 of the first portion 402 may be or describe one or more portions or regions (e.g., end, edge, middle, etc.) of the first portion 402 that is selectively movable relative to a bone or bone structure. For example, where a synthetic graft, an allograft, and/or autograft is implemented, a central portion of a graft (e.g., the first portion 402) may be looped around a bone or bone structure (e.g., a coracoid process 122, etc.) to secure the graft to the bone, while leaving the ends of the graft movable. In such instances, the central portion of the graft (e.g., a central region of the first portion 402 that is looped around the coracoid process 122, etc.) may be referred to as the first end 152, while the two ends of tissue that are movable may be referred to as the second end 410 (e.g., two second ends 410, etc.). Similarly, in another instance where a synthetic, an allograft, and/or autograft is implemented, a central portion of a graft (e.g., the first portion 402, etc.) may be folded and/or driven into a hole in a bone or bone structure (e.g., the glenoid rim 126 of the scapula 104, an anterior and/or medial portion of the glenoid of the scapula 104, etc.) to secure the graft to the bone, while leaving one or more ends of the graft movable. In such instances, the central portion of the graft (e.g., a central region of the first portion 402 that is folded/driven into the hole in the bone, etc.) may be referred to as the first end 152, while the two ends of tissue that are movable may be referred to as the second end 410 (e.g., two second ends 410, etc.). All such embodiments and/or configurations are contemplated herein.

As shown in FIGS. 4A-4B, in both exemplary variations, the second portion 404 may be coupled to a bone. For example, as part of the tenodesis, the first end 412 of the second portion 404 may be coupled to the humerus 106 (e.g., after the biceps 150 has been modified or cut, etc.). In exemplary embodiments, the first end 412 of the second portion 404 is coupled to the humerus 106 using an interlacing of a tissue junction (e.g., tissue of the first end 412, etc.) with an elongated tensionable structure or wire, and/or coupling (e.g., anchoring, fixing, connecting, etc.) the first end 412 to the humerus 106 via an anchor, shown as anchor 414. In exemplary embodiments, the elongated tensionable structures and/or anchors used to anchor the first end 412 to the humerus 106 are the same and/or similar to other elongated tensionable structures and/or anchors described elsewhere herein.

As also shown in FIGS. 4A-4B, in both illustrated examples, the first portion 402 may be manipulated relative to one or more features of the shoulder 100. For example, the second end 410 (e.g., free end, etc.) of the first portion 402 may be manipulated (e.g., moved, repositioned, etc.) into and/or through a joint space of the shoulder 100 (e.g., an intraarticular portion, etc.). In some embodiments, and as described elsewhere herein, the second end 410 (e.g., free end, etc.) of the first portion 402 is manipulated using one or more elongated tensionable structures or the like connected with the first portion 402, one or more surgical devices and/or tools (e.g., a grasper, etc.), via the assistance of one or more surgical devices and/or tools (e.g., a camera, etc.), and/or using one or more suitable techniques (e.g., arthroscopically, arthroscopically through a portal, non-arthroscopically, etc.).

Referring now to FIGS. 5A-5B, examples of manipulations of the first portion 402 are shown. Specifically, the processes illustrated in FIGS. 5A-5B may include one or more preparation processes. According to exemplary embodiments, the process illustrated in FIG. 5A may include the subpectoral preparation technique of FIG. 4A and the process illustrated in FIG. 5B may include the suprapectoral preparation technique of FIG. 4B. As shown in FIGS. 5A-5B, in both exemplary variations, the second end 410 (e.g., free end, etc.) of the first portion 402 may be manipulated (e.g., moved, repositioned, etc.) into and/or through a joint space of the shoulder 100. In exemplary embodiments, the second end 410 is prepared (e.g., for manipulation, anchoring, attachment, coupling, etc.). For example, the second end 410 may be prepared by coupling the second end 410 with one or more elongated tensionable structures, shown as elongated tensionable structures 502. In some embodiments, the second end 410 of the first portion 402 is interlaced with elongated tensionable structures (e.g., the elongated tensionable structures 502, etc.), for example in preparation for anchoring the second end 410 (e.g., to a bone, etc.). The elongated tensionable structures 502 may be the same as or similar to one or more elongated tensionable structures described herein, and such interlacing may include whipstitching the second end 410, using Krackow stitching, and/or another suitable technique, as known in the art.

In some embodiments, one or more characteristics of the second end 410 are collected, analyzed, and/or modified. For example, a diameter of the second end 410 may be measured (e.g., with a block, a sizing block, etc.). Further, in some embodiments, the second end 410 may be modified and/or adjusted. For example, the second end 410 may be trimmed (e.g., cut, etc.), for example to a predetermined diameter (e.g., 4 mm, 4.5 mm, 5 mm, 5.5 mm, 6 mm, 6.5 mm, 7 mm, etc.) to accommodate for manipulating, routing, anchoring, and/or securing the second end 410. Certain embodiments of techniques described below are illustrated using first portion 402 as prepared in FIG. 4A (e.g., the subpectoral technique, etc.), which as noted above may result in a longer first portion 402 length. This longer length may provide the surgeon with more graft material to allow for the various positioning and routing details exemplified by the following illustrated techniques. It should be understood that the techniques described below may also be implemented with the first portion 402 as prepared in FIG. 4B (e.g., the suprapectoral technique, etc.), which as noted above may result in a shorter first portion 402 length. In some instances, elongated tensionable structures and/or materials may be attached to the first portion 402 to extend the overall length of the graft material (e.g., an overall length of the first portion and the elongated tensionable structures or filaments, etc.), which may allow for various positioning and/or routing details exemplified by the following illustrated techniques. All such combinations and variations are contemplated herein.

For example, and as shown in FIG. 5A, the processes described herein may be implemented with the biceps 150 being modified via a subpectoral technique (e.g., as discussed in FIG. 4A). Further, and as shown in FIG. 5B, it is contemplated that the processes described herein may also be implemented with the biceps 150 being modified via the suprapectoral technique (e.g., as discussed in FIG. 4B), and/or implementations thereof (e.g., including elongated tensionable structures and/or materials extending from a cut end to provide a longer overall length of the graft/material, etc.). All such combinations and variations are contemplated herein.

Referring still to FIGS. 5A-5B, the processes may include preparing one or more bones, according to exemplary embodiments. For example, a tunnel, groove, guide, or bore hole may be prepared in a bone. In exemplary embodiments, a tunnel, shown as tunnel 504, is prepared in the clavicle 102. The tunnel 504 may be prepared using one or more surgical devices described herein (e.g., a guidewire, a drill, etc.). In some embodiments, the tunnel 504 has a predetermined diameter (e.g., 4 mm, 4.5 mm 5 mm, 5.5 mm, 6 mm, 6.5 mm, 7 mm, etc.). For example, and as will be described herein, a diameter of the tunnel 504 may be equal to or larger than a diameter of the second end 410 to facilitate moving the second end 410 to and/or through the tunnel 504.

In some embodiments, the tunnel 504 is prepared with one or more components or devices (e.g., prosthetic devices, etc.). For example, the tunnel 504 may be prepared to include a protective layer or component to protect one or more tissues or components received therein (e.g., a tendon, tissue, elongated tensionable structure, etc.) from damage (e.g., wear and tear, severing, degradation, etc.), for example from engaging with an adjacent structure (e.g., a bone surface, an edge of a tunnel through a bone, etc.). In some embodiments, the protective layer or component is a sheath (e.g., a synthetic sheath, a protective sheath, etc.), a clip, a tube, a guide tube, and/or another suitable protective component.

In some embodiments, a bone is prepared to include one or more tunnels, channels, grooves, guides, or holes. For example, and in the exemplary embodiments of FIGS. 5A-5B, the clavicle 102 is prepared to include a first tunnel (e.g., the tunnel 504, etc.) and a second hole or tunnel (e.g., to receive the first anchor 302, to receive the elongated tensionable structure 306, etc.). In this regard, the clavicle 102 may be prepared to include the tunnel 504 and a second hole or tunnel (e.g., to receive the first anchor 302, the elongated tensionable structure 306, etc.) to facilitate the tensioning processes and a stabilization processes, as described herein. To ensure stability of the clavicle 102 (e.g., structural stability, structural integrity, etc.), the first tunnel (e.g., the tunnel 504, etc.) and the second hole (or tunnel) may be spaced a predetermined distance apart.

In other embodiments, a bone is prepared to include a single tunnel, channel, groove, guide, or hole. For example, in some embodiments the clavicle 102 is prepared to include a tunnel (e.g., the tunnel 504, etc.), which may be configured to receive one or more components described herein (e.g., the elongated tensionable structure 306, the first anchor 302, the second end 410 of the first portion 402, etc.). For example, the clavicle 102 may be prepared to include the tunnel 504 (e.g., which may or may not include a sheath, a plate, a clip, etc. as described herein). The tunnel 504 may be configured to receive the second end 410 (e.g., of the biceps 150) and a component for tensioning the shoulder 100 (e.g., the first anchor 302, the elongated tensionable structure 306, etc. described herein), for example to facilitate both the tensioning processes and a stabilization processes, as described herein. In this regard, in some embodiments the tunnel 504 may be configured to receive the second end 410 and a component for tensioning the shoulder 100 to reduce the number of holes in the first bone (e.g., the clavicle 102, etc.), thereby reducing the risk of fractures.

In some embodiments, the processes may include preparing one or more components of a joint, according to example embodiments. For example, in some instances, the tissue may be removed and/or attached (e.g., reattached, etc.). This may be done for a variety of reasons, for example the tissue being too short, a desire to reinforce the joint or shoulder structure (e.g., an anterior labrum, etc.), and/or a surgeon or procedural preference (e.g., due to tissue characteristics, for stability and/or reinforcement purposes, etc.). For example, in instances where the biceps 150 (e.g., the first end 152, etc.) exhibits trauma or damage (e.g., a tearing or ripping at the first end 152, a tearing or ripping from the tubercle 124, a structure at the glenoid cavity, etc.) or is desired to be shifted, the biceps 150 may be removed and/or attached (e.g., reattached, etc.) to a component of the shoulder 100 (e.g., the scapula 104, etc.). For example, the biceps 150 (e.g., the first end 152) may be removed from the scapula 104 (e.g., at the tubercle 124, etc.), and may be attached (e.g., reattached, etc.) to the scapula 104. In this regard, and as described elsewhere herein, the biceps 150 (e.g., the first end 152, etc.) may be reattached to a bone or bone structure (e.g., of the scapula 104, etc.) at the same native location (e.g., at the tubercle 124, etc.) and/or another location (e.g., the glenoid rim 126, the coracoid process 122, or other locations at the scapula 104, etc.).

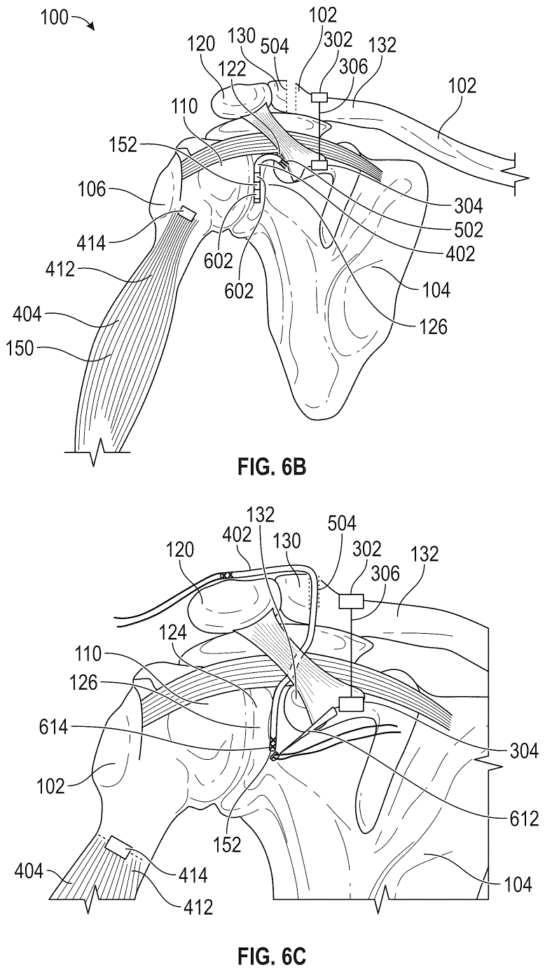

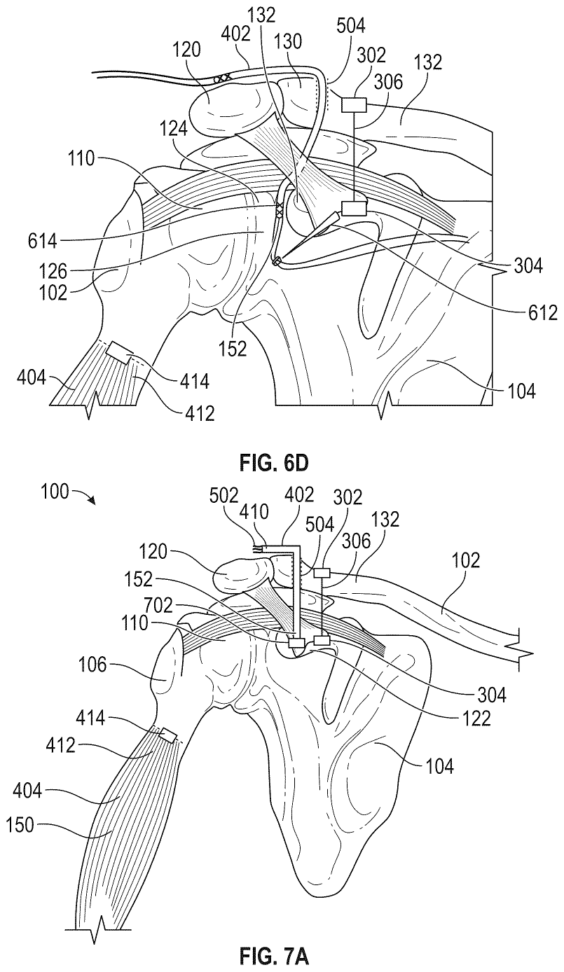

In some embodiments, and as shown in FIGS. 6A-6B, the first end 152 may be attached to a bone or bone structure of the scapula 104 (e.g., a bone or bone structure around the glenoid cavity of the scapula 104, etc.). For example, the first end 152 may be attached to the glenoid rim 126 in a vertical or semi vertical configuration. In other embodiments, the first end 152 is attached to another portion of the scapula 104 (e.g., adjacent the glenoid rim 126, at the coracoid process 122, etc.), and/or in another suitable configuration (e.g., at a 5, 10, 15, 30, 45, 50, 60, 90, etc. degree angle relative to the glenoid rim 126, etc.). As shown, the first end 152 may be attached to the glenoid rim 126 via one or more anchors or stabilizing members, shown as anchors 602. The anchor 602 may be a pin or screw (e.g., an interference screw, a top hat button, a drop crotch button, etc.), an elongated tensionable structure or stitching, which may be the same as or similar to other anchors and/or elongated tensionable structures described herein (e.g., for an inlay and/or onlay technique, etc.).

As shown in FIG. 6A, it should be understood that any and/or all of the processes described herein may be implemented with the biceps 150 being modified via a subpectoral technique (e.g., as discussed in FIG. 4A). Further, and as shown in FIG. 6B, it is also contemplated that the processes described herein may be implemented with the biceps 150 being modified via the suprapectoral technique (e.g., as discussed in FIG. 4B), and/or implementations thereof (e.g., including elongated tensionable structures, filaments, and/or materials extending from a cut end to provide a longer overall length of the graft/material, etc.). All such combinations and variations are contemplated herein.

In some embodiments, and as shown in FIGS. 6C-6D, the first end 152 may be attached to the glenoid rim 126 via one or more knotless anchors, shown as anchors 612, as described elsewhere herein. As described herein, the first end 152 may be attached (e.g., to the glenoid rim 126) using one or more suitable techniques. For example, and as shown in FIG. 6C, the first end 152 may be secured (e.g., attached, coupled, etc.) to the scapula 104 (e.g., the glenoid rim 126, etc.) using an inlay technique, in which the anchors 612 are used to secure at least a portion of the first end 152 within an anchor hole to secure the first end 152 relative to the glenoid rim 126. In other embodiments, and as shown in FIG. 6D, the first end 152 may be secured (e.g., attached, coupled, etc.) to the scapula 104 (e.g., the glenoid rim 126, etc.) using an onlay technique, in which the anchors 612 (or no anchors) are used to secure at least a portion of the first end 152 to a surface of the glenoid rim 126. In yet other embodiments, additional stabilization features (e.g., elongated tensionable structures, anchors, stitching, etc.) may be used to further secure (e.g., attach, couple, etc.) a graft and/or tissue (e.g., the first end 152, etc.) to a portion of the scapula 104 (e.g., a portion of the glenoid, etc.), for example for increased stability, as described herein.

As described herein, an “inlay” technique may refer to a technique where a tissue and/or augment is connected (e.g., anchored, secured, attached, etc.) to a bone by driving the tissue and/or augment into an anchor hole. As also described herein, an “onlay” technique may refer to a technique where a tissue and/or augment is connected (e.g., anchored, secured, attached, etc.) to a bone without driving the tissue and/or augment into an anchor hole.

For example, and as shown in FIG. 6C, in an inlay technique, a tool may be used to manipulate (e.g., pull, push, force, etc.) tails (e.g., one or more tails of an elongated tensionable structure that is whipstitched, shown as stitching 614, to a tissue or graft, shown as the first end 152, etc.) into a hole or channel in a bone (e.g., a hole or channel in the glenoid rim 126, the scapula 104, etc.). As the tails are manipulated, a portion of tissue (e.g., a portion of the first end 152, etc.) may be drawn (e.g., forced, pushed, pulled, etc.) into the hole, thereby securing and/or stabilizing the tissue (e.g., the first end 152, etc.) relative to the bone (e.g., the glenoid rim 126, the scapula 104, etc.). In some embodiments, the tissue (e.g., the first end 152, etc.) is received in the hole (e.g., the hole in the glenoid rim 126, the scapula 104, etc.), and secured (e.g., anchored, coupled, etc.) using one or more anchors (e.g., the anchor 612, etc.), as described elsewhere herein. In other embodiments, the tissue (e.g., the first end 152, etc.) is received in the hole (e.g., the hole in the glenoid rim 126, the scapula 104, etc.), and secured (e.g., anchored, coupled, etc.) without use of an anchor.

In other embodiments, and as shown in FIG. 6D, in an onlay technique, a tool may be used to manipulate (e.g., pull, push, force, etc.) tails (e.g., one or more tails of an elongated tensionable structure that is whipstitched, shown as stitching 614, to a tissue or graft, shown as the first end 152, etc.) into a hole or channel in a bone (e.g., a hole or channel in the glenoid rim 126, the scapula 104, etc.). As the tails are manipulated, a portion of tissue (e.g., a portion of the first end 152, etc.) may be drawn (e.g., forced, pushed, pulled, etc.) toward the hole. With the tissue (e.g., the first end 152, etc.) positioned at or adjacent to the hole, the tails may be received in the hole and/or secured (e.g., anchored, coupled, etc.) using one or more anchors (e.g., the anchor 612, etc.), as described herein. In some embodiments, with the tissue (e.g., the first end 152, etc.) positioned at or adjacent to the hole, the tissue (e.g., the first end 152, etc.) may be secured to one or more bones or bone structures (e.g., the glenoid rim 126, the scapula 104, etc.), for example using one or more elongated tensionable structures (e.g., whipstitching, a Krackow stitching, etc.), as described elsewhere herein. In this regard, in some embodiments, the tissue (e.g., the first end 152, etc.) may be connected to one or more bones or bone structures (e.g., the glenoid rim 126, the scapula 104, etc.) using a techniques (e.g., onlay technique, etc.) which does not utilize an anchor.

It should also be understood that while FIGS. 6C-6D illustrate the biceps 150 as being modified via a subpectoral technique (e.g., as discussed in FIG. 4A), it is contemplated that the processes illustrated in FIGS. 6C-6D may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

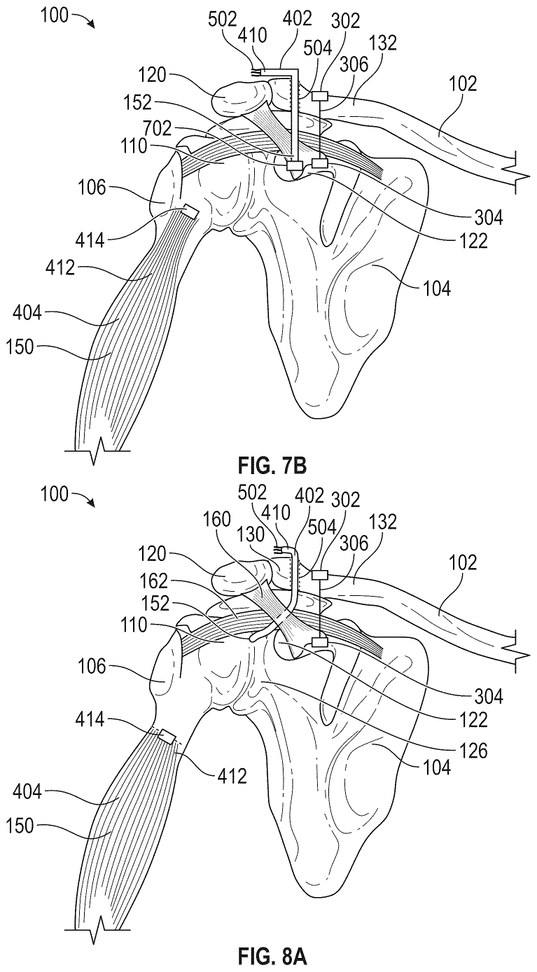

In other instances, and as described elsewhere herein, the biceps 150 (e.g., the first portion 402, etc.) may be attached to another suitable bone or bone structure. For example, and as shown in FIGS. 7A-7B, the first end 152 may be attached to the coracoid process 122 (e.g., an inferior portion of the coracoid process 122, etc.). The first end 152 may be attached to the coracoid process 122 via an anchor or stabilizing member, shown as anchor 702. The anchor 702 may be a pin or screw (e.g., an interference screw, a tenodesis screw, a knotless anchor, etc.), or another suitable anchor (e.g., knotted, knotless, all-suture, PEEK, buttons, plate, etc.), which may be the same as or similar to other anchors described herein. In some instances, the first end 152 may be attached (e.g., reattached, etc.) to the scapula 104 at the same or a different location from where the first end 152 was removed (e.g., via the anchor 702, etc.). Further, and as described elsewhere herein, it should be understood that any and/or all of the processes described herein may be implemented with the biceps 150 being modified via a subpectoral technique (e.g., as shown in FIG. 7A), a suprapectoral technique (e.g., as shown in FIG. 7B), and/or implementations thereof (e.g., including elongated tensionable structures, filaments, and/or materials extending from a cut end to provide a longer overall length of the graft/material, etc.). All such embodiments are contemplated herein.

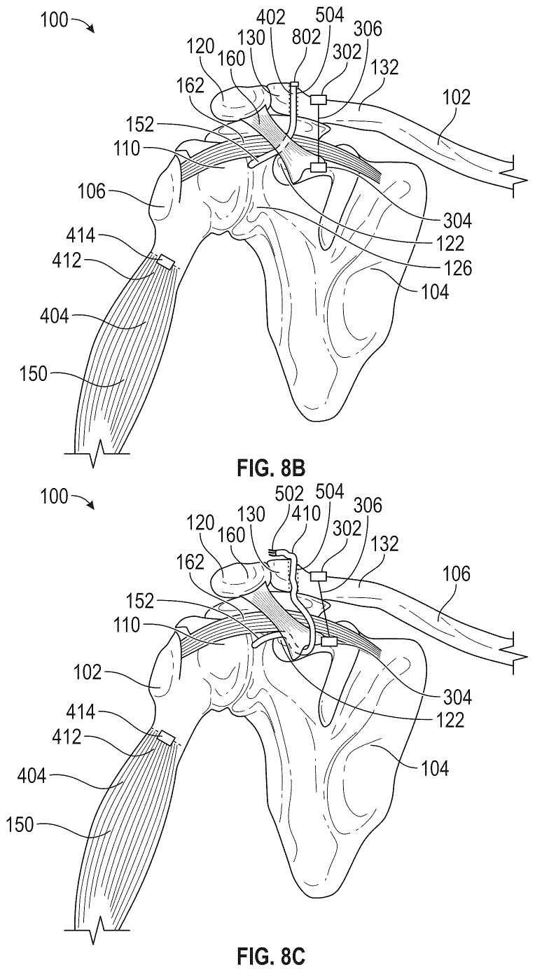

Referring now to FIGS. 8A-10B, the various aspects described herein may include one or more guidance, movement, and/or retrieval processes, according to exemplary embodiments. In exemplary embodiments, the second end 410 of the first portion 402 may be manipulated (e.g., moved, repositioned, etc.) into and/or through the tunnel 504. For example, the second end 410 of the first portion 402 may be manipulated (e.g., moved, repositioned, etc.) from an inferior position (e.g., an inferior portion of the clavicle 102, etc.), into and/or through the tunnel 504 to a superior position (e.g., a superior portion of the clavicle 102, etc.). In some embodiments, the second end 410 is manipulated (e.g., moved, shuttled, etc.) to and/or through the tunnel 504 via one or more surgical devices or tools (e.g., a guidewire, an elongated tensionable structure, etc.). A force may be applied to the second end 410, for example to stabilize the second end 410 and/or the first portion 402 in predetermined condition (e.g., in tension, etc.).

In some embodiments, the one or more guidance, movement, and/or retrieval processes includes the second end 410 of the first portion 402 being manipulated (e.g., moved, repositioned, etc.) relative to one or more components of a shoulder (e.g., the shoulder 100, etc.), as described herein. For example, the second end 410 may be manipulated relative to a ligament, tissue, bone and/or bone structure of the shoulder 100.

As shown in FIG. 8A, the second end 410 may be manipulated relative to the coracoacromial ligament 160 and/or the tissue 162 (e.g., the supraspinatus muscle, etc.). For example, the second end 410 (e.g., the first portion 402, etc.) may be manipulated such that the second end 410 is routed posteriorly relative to some tissue (e.g., the coracoacromial ligament 160, etc.) and/or anterior/superiorly relative to some tissue (e.g., the tissue 162, the supraspinatus muscle, etc.). In some embodiments, the second end 410 is manipulated relative to the coracoacromial ligament 160 and/or the tissue 162 (e.g., the supraspinatus muscle, etc.), for example such that the second end 410 is otherwise routed (e.g., relative to the coracoacromial ligament 160, the tissue 162, the supraspinatus muscle, etc.).

As discussed herein, the first portion 402 may also be secured relative to one or more ligaments, tissues, bones, and/or bone structures of the joint. As shown in FIG. 8B, with the first portion 402 manipulated into and/or through the tunnel 504, the first portion 402 is secured with the clavicle 102. According to an exemplary embodiment, securing the first portion 402 with the clavicle 102 may provide stabilization to the shoulder 100 and/or joint space (e.g., vertical stability to the clavicle, vertical stability to the AC joint, etc.).

As shown, the first portion 402 is secured to the clavicle 102 and/or within the tunnel 504 via an anchor or stabilizing anchor, shown as anchor 802. In exemplary embodiments, the anchor 802 is an interference screw that is driven into the tunnel 504 to secure the first portion 402 within the tunnel 504. For example, the anchor 802 may be an interference screw that is driven into the tunnel 504 to secure elongated tensionable structures attached to the first portion 402 (e.g., the elongated tensionable structures 502 shown in at least FIG. 8B, etc.) between threads of the anchor 802 and one or more sides of the tunnel 504. In other embodiments, the anchor 802 is an interference screw that is driven into the tunnel 504 to secure the first portion 402 (e.g., within the tunnel 504, as shown in at least FIG. 8B, etc.) by compressing at least a portion of the first portion 402 between the threads of the anchor 802 and the side of the bone tunnel 504.

It should be understood that while the anchor 802 is described as being an interference screw, it is contemplated that the anchor 802 may be any suitable anchor described elsewhere herein, including for example a knotless anchor, an all-elongated tensionable structure anchor, a button anchor, and/or a tenodesis screw, or the like. In this sense, it is contemplated that the anchor 802 may be another suitable anchor that is used in the manner of an interference screw (i.e., by compressing at least a portion of the first portion 402 between the threads of the anchor 802 and the side of the bone tunnel 504). Further, it should also be understood that while FIGS. 8A-8B illustrate the biceps 150 as being modified via a subpectoral technique (e.g., as discussed in FIG. 4A), it is contemplated that the processes illustrated in FIGS. 8A-8B may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

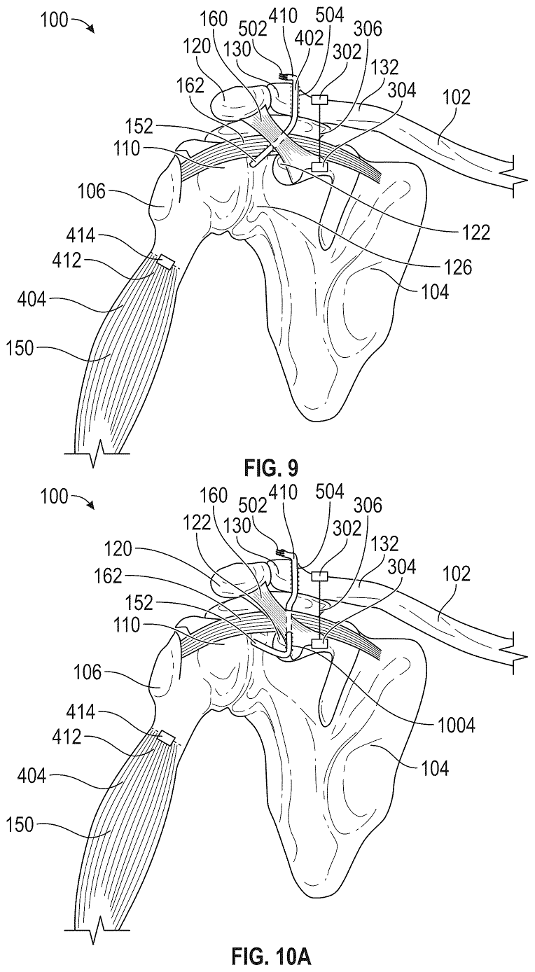

As shown in FIG. 8C, the second end 410 may also be manipulated relative to the coracoid process 122 (e.g., a posterior portion, an interior portion, an anterior portion, etc. of the coracoid process 122). For example, the second end 410 (e.g., the first portion 402, etc.) may be manipulated such that the second end 410 is routed relative to the coracoid process 122 (e.g., under the coracoid process 122, etc.), then anteriorly relative to the coracoid process 122, for example to be routed around the coracoid process 122 (e.g., looped around, etc.). The second end 410 may further be routed relative to the coracoacromial ligament 160 and/or the tissue 162 (e.g., the supraspinatus muscle, etc.), for example as discussed with reference to FIG. 8A. In other embodiments, the second end 410 is otherwise routed relative to one or more ligaments, tissues, bones, and/or bone structures (e.g., to and/or through the coracoacromial ligament 160, the tissue 162 or the supraspinatus muscle, etc.), for example as will be discussed with reference to FIG. 9. It should be understood that while FIG. 8C illustrates the biceps 150 as being modified via a subpectoral technique (e.g., as discussed in FIG. 4A), it is contemplated that the process illustrated in FIG. 8C may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

In some embodiments, the second end 410 is further routed relative to one or more ligaments, tissues, bones, and/or bone structure. For example, and as shown in FIG. 9, the second end 410 (e.g., the first portion 402, etc.) may be routed through the tissue 162, such that the second end 410 (e.g., the first portion 402, etc.) moves through a center or belly of the tissue 162 (e.g., a belly or center of the supraspinatus muscle, etc.). In some instances, a tunnel or pathway is created and/or formed in the tissue 162 (e.g., the supraspinatus muscle, etc.), such that the second end 410 of the first portion 402 may be manipulated to and/or through the tissue 162 (e.g., the supraspinatus muscle, etc.), as shown in FIG. 9. It should be understood that while FIG. 9 illustrates the biceps 150 as being modified via a subpectoral technique (e.g., as discussed in FIG. 4A), it is contemplated that the process illustrated in FIG. 9 may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

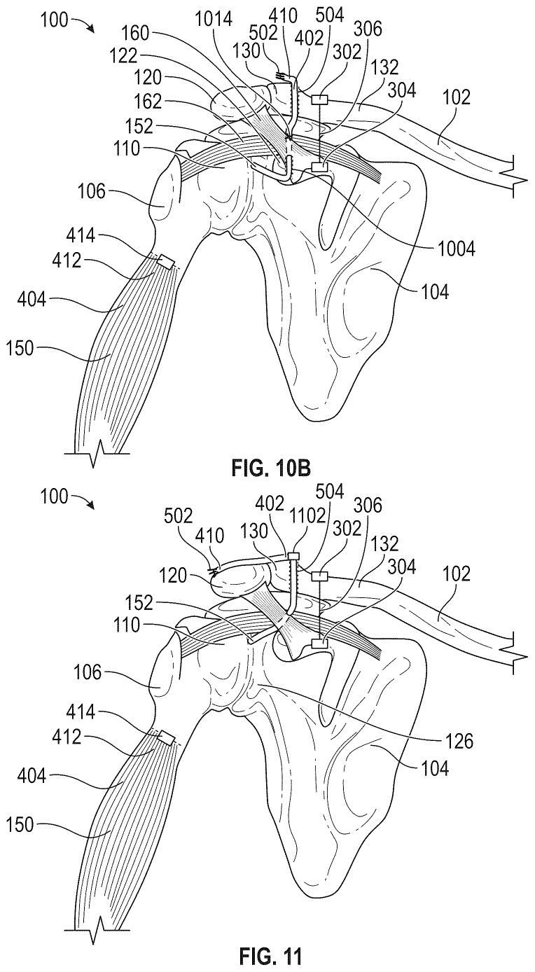

In other embodiments, the second end 410 is yet further or otherwise routed relative to one or more ligaments, tissues, bones, and/or bone structures of the shoulder 100. For example, and as shown in FIGS. 10A-10B, the second end 410 may be routed through a bone or bone structure (e.g., the coracoid process 122, etc.). In example embodiments, the second end 410 (e.g., the first portion 402, etc.) is routed through a tunnel (e.g., shown as tunnel 1004) in the coracoid process 122, for example from an inferior position (e.g., an inferior portion of the coracoid process 122, etc.), into and/or through the tunnel 1004 to a superior position (e.g., a superior portion of the coracoid process 122, etc.). As shown in FIG. 10A, the second end 410 may further be routed relative to one or more features of the joint (e.g., the coracoacromial ligament 160, the tissue 162 or the supraspinatus muscle, through the tissue 162 or the supraspinatus muscle, etc.), as described herein.

As discussed herein, in some embodiments, the first portion 402 may be secured relative to one or more ligaments, tissues, bones, and/or bone structures of the joint. For example, and as shown in FIG. 10B, the first portion 402 may be secured to the coracoacromial ligament 160. In example embodiments, the first portion 402 is secured to the coracoacromial ligament 160 via an elongated tensionable structure or stitching, shown as stitching 1014, which may be the same as or similar to other elongated tensionable structures and/or stitching, described herein. In other embodiments, the first portion 402 is secured to the coracoacromial ligament 160 via an anchor or another suitable stabilizing structure, as described herein. In other embodiments, the first portion 402 may be otherwise secured to another tissue or bone structure (e.g., a portion of the coracoacromial ligament 160, a portion of the coracoacromial ligament 160 not shown, etc.), as also described elsewhere herein. It should be understood that while FIGS. 10A-10B illustrate the biceps 150 as being modified via a subpectoral technique (e.g., as discussed in FIG. 4A), it is contemplated that the processes illustrated in FIGS. 10A-10B may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

It should be understood that any and/or all of the preparation, guidance, movement, and/or retrieval processes described herein (e.g., the preparation, guidance, and/or movement processes described with reference to FIGS. 6A-10B) may be combined and/or implemented in any suitable combination. For example, it is contemplated that in one exemplary instance, the first end 152 may be attached to the glenoid rim 126 (e.g., via an inlay technique as shown in FIG. 6C, or an onlay technique as shown in FIG. 6D, etc.), and the second end 410 routed around the coracoid process 122 (e.g., as shown in FIG. 8C, etc.) or through the coracoid process 122 (e.g., as shown in FIG. 10A), through the tissue 162 (e.g., through a medial portion of the supraspinatus tissue, for example similar to as illustrated in FIG. 9), and to/through the tunnel 504. Further, with the second end 410 positioned through the tunnel, the first portion 402 may be secured relative to one or more portions of the coracoacromial ligament, for example for added stability and/or stabilization. All such combinations of embodiments are contemplated herein.

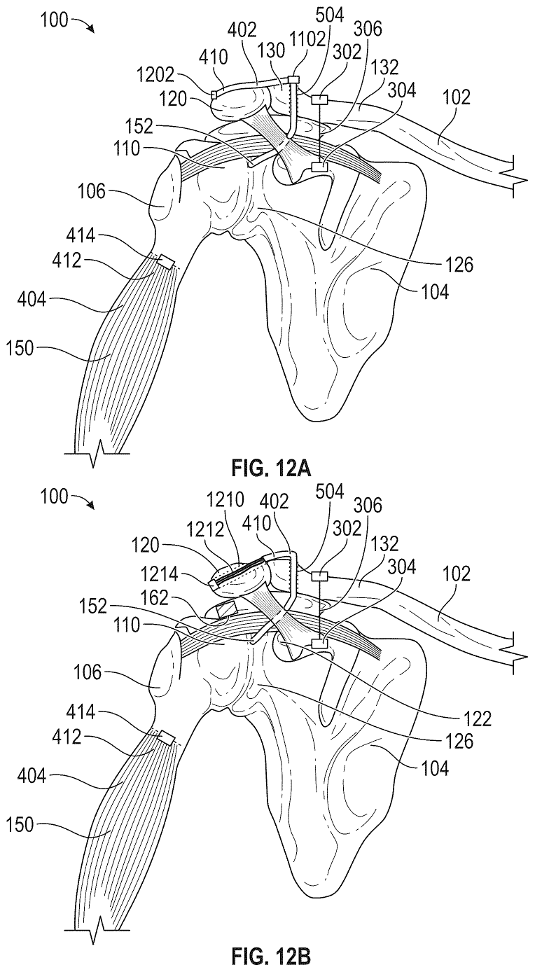

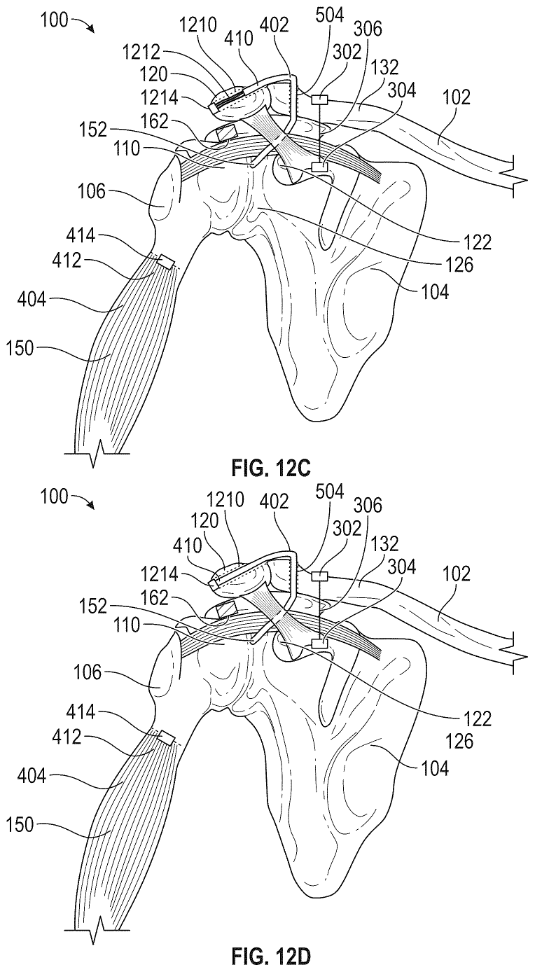

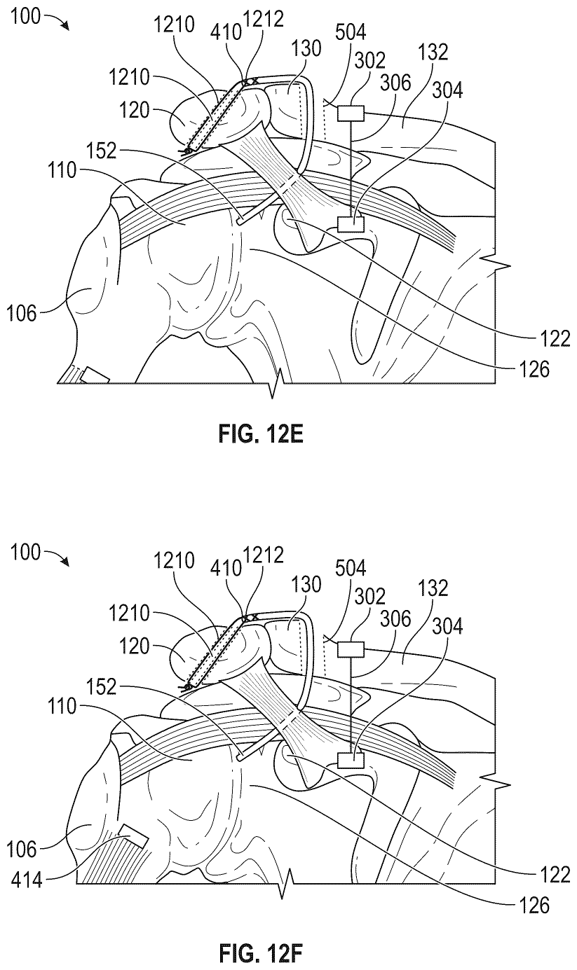

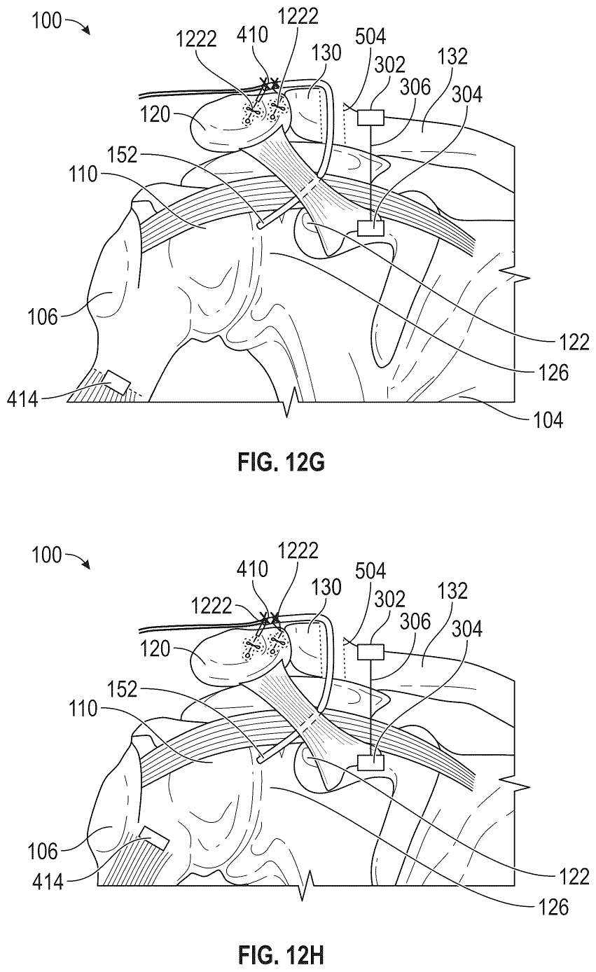

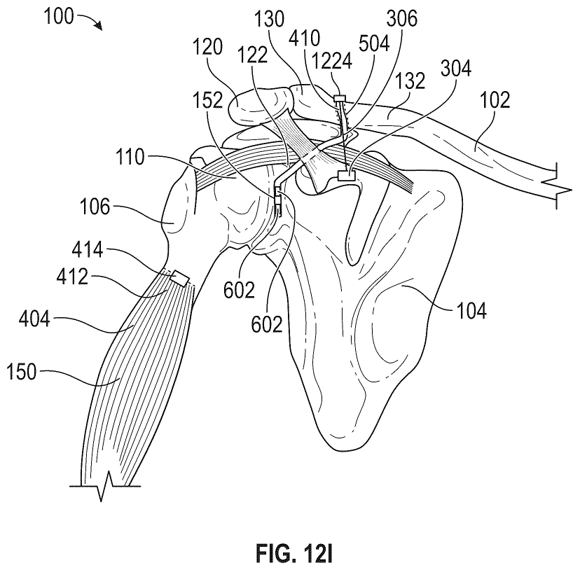

Referring now to FIGS. 11-12I, the various processes described herein may include one or more stabilization processes, according to exemplary embodiments. As shown, with the second end 410 manipulated to and/or through the tunnel 504, the second end 410 may be manipulated (e.g., moved, repositioned, guided, etc.) across one or more bones. For example, the second end 410 may be manipulated (e.g., moved, repositioned, guided, etc.) across the clavicle 102 and/or the scapula 104 (e.g., along the clavicle 102, across the AC joint, etc.), for example to a connection or anchor point at a bone (e.g., the acromion 120 of the scapula 104, etc.). In exemplary embodiments, the second end 410 is manipulated (e.g., moved, repositioned, guided, etc.) across one or more components of the shoulder 100 (e.g., the clavicle 102, the scapula 104, the AC joint, etc.), for example to provide stabilization over and/or around the shoulder 100 and/or a joint space (e.g., vertical stability to the clavicle 102, vertical stability to the clavicle 102 relative to the acromion 120, vertical stability to the AC joint, etc.).

In some embodiments, the second end 410 is manipulated (e.g., moved, repositioned, guided, etc.) across one or more bones that include a component or device. For example, in implementations where a bone (e.g., the clavicle 102, etc.) includes a plate or implant (e.g., a hook plate, etc.), the second end 410 may be manipulated (e.g., moved, positioned, etc.) relative to the plate or implant. In exemplary embodiments, the second end 410 is positioned at an opening or gap in the plate or implant (e.g., an opening in an end or side of a hook plate, an opening in an end or side of an elongated plate, etc.)to facilitate removal of the plate or implant, without altering the positioning and/or placement of the second end 410 (e.g., the first portion 402, etc.) relative to the bone (e.g., the clavicle 102, etc.).

As shown in FIG. 11, with the second end 410 of the first portion 402 in a condition (e.g., in tension, a determined condition, a predetermined condition, a desired condition, etc.), an anchor or stabilizing anchor, shown as anchor 1102, may be secured (e.g., attached, coupled, etc.) with the first portion 402. The anchor 1102 may be a pin or screw (e.g., an interference screw, etc.), which may be the same as or similar to other anchors described herein. For example, the anchor 1102 may be a hard body anchor, according to various aspects. In exemplary embodiments, the anchor 1102 is secured to the first portion 402 and received by the tunnel 504, for example to stabilize (e.g., secure, anchor, etc.) the first portion 402 within the tunnel 504 and/or relative to a bone (e.g., the clavicle 102, etc.). The remainder of the first portion 402, extending beyond anchor 1102, may be removed or otherwise further fixated.

In some embodiments, for example as illustrated in FIG. 12, this extending portion of first portion 402 may be secured at a lateral location via anchor 1202, as discussed in greater detail below. It should be understood that while FIG. 11 illustrates the biceps 150 as being modified via a subpectoral technique (e.g., as discussed in FIG. 4A), it is contemplated that the process illustrated in FIG. 11 may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

As shown in FIGS. 12A-12I, with the second end 410 (e.g., the first portion 402, etc.) manipulated to and/or through the tunnel 504, the first portion 402 (e.g., the second end 410, etc.) may be secured (e.g., fixated, anchored, stabilized, coupled, etc.) to a bone. For example, the second end 410 may be manipulated to a connection or anchor point, for example on the scapula 104 (e.g., the acromion 120, etc.). Further, the second end 410 may be secured (e.g., fixed, fixated, anchored, stabilized, coupled, etc.) to the anchor point (e.g., the acromion 120, the scapula 104, etc.). In this regard, the first portion 402 may be manipulated such that the first portion 402 is routed through the clavicle 102 (e.g., through the tunnel 504, etc.), along a superior surface of the clavicle 102, over and/or across a joint space to the scapula 104, and secured (e.g., anchored, attached, coupled, etc.) to an anchor point at the acromion 120, for example to provide increased stability to a joint (e.g., the shoulder 100, etc.).

As described herein, it should be understood that the second end 410 may be secured (e.g., attached, interface, engage, anchored, fixed, fixated, coupled, etc.) to a bone (e.g., the scapula 104, etc.) using any suitable technique. For example, and as shown in FIG. 12A, an anchor or retention anchor, shown as anchor 1202, may secure (e.g., couple, attached, fixated, etc.) the first portion 402 (e.g., the second end 410, etc.) to a bone. As described herein, the second end 410 may be secured within an anchor hole in a bone via the anchor 1202, for example when implemented using an inlay technique. Further, the second end 410 may be secured along a surface of a bone, shown as the acromion 120, for example when implemented using an onlay technique. In some embodiments, the anchor 1202 is a knotless anchor, button anchor, or the like, and is configured to receive one or more elongated tensionable structures (e.g., the elongated tensionable structures 502, etc.), extending from the second end 410 to secure the elongated tensionable structures to the bone. Such elongated tensionable structures may extend into the bone hole along with the anchor (e.g., an inlay technique), or may be positioned and secured relative to the anchor above the bone hole into which the anchor may be situated (e.g., an onlay technique).

Alternatively, the first portion 402 itself can be positioned with the anchor 1202 into the bone, commonly known as a tenodesis technique, and thus the elongated tensionable structures associated with the first portion 402 (if any) may be used to manipulate the second end 410 into position for fixation, further stabilize the attachment, and/or may be trimmed and removed at some time during the procedure. In another alternative, the first portion 402, for example the second end 410, may be secured to an anchor positioned in bone though the first portion 402 itself may remain proud of the bone. In some embodiments, the anchor 1202 is the same as or similar to anchors described herein.

As discussed elsewhere herein, it should be understood that while FIG. 12A illustrates the biceps 150 as being modified via a subpectoral technique (e.g., as discussed in FIG. 4A), it is contemplated that the process illustrated in FIG. 12A may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

In some embodiments, the second end 410 and/or the first portion 402 is otherwise secured to a bone (e.g., the acromion 120, the scapula 104, etc.). For example, and as shown in FIGS. 12B-12F, one or more tunnels, shown as tunnel 1210, may be created in the bone (e.g., the acromion 120, the scapula 104, etc.).

As shown in at least FIGS. 12B-12D, in some embodiments one tunnel 1210 is created in the acromion 120. According to exemplary embodiments, the tunnel 1210 extends through the acromion 120 from a superior portion, toward and/or through a lateral portion of the acromion 120. In other embodiments, the tunnel 1210 is otherwise arranged or configured, including, for example extending from a superior portion to an inferior portion of the acromion 120, from a superior portion to/out of an anterior side of the acromion 120, and/or another suitable configuration.

As shown in FIGS. 12B-12D, the tunnel 1210 is configured to receive one or more portions of a ligament, tissue, elongated tensionable structure, and/or anchor, as described herein. For example, and as shown in at least FIG. 12B, elongated tensionable structures, shown as lead elongated tensionable structures 1212, may be attached to and/or extend from the second end 410 of the first portion 402. As described herein, the lead elongated tensionable structures 1212 may be elongated tensionable structure tails extending from whipstitching at the second end 410. The lead elongated tensionable structures 1212 may be manipulated through the tunnel 1210, such that the lead elongated tensionable structures 1212 extend through the tunnel 1210 and/or to an end of the tunnel 1210 to a lateral side of the acromion 120.

As shown in at least FIG. 12B, the lead elongated tensionable structures 1212 are secured to a lateral side of the acromion 120 at an end of the tunnel 1210 via an anchor or stabilizing anchor, shown as anchor 1214. As described elsewhere herein, in exemplary embodiments the anchor 1214 may be an interference screw that is driven into the tunnel 1210 to secure the first portion 402 relative to the acromion 120. For example, the anchor 1214 may be an interference screw that is driven into the tunnel 1210 to secure the lead elongated tensionable structures 1212 between the threads of the anchor 1214 and one or more sides of the tunnel 1210. In other embodiments, the anchor 1214 is another suitable anchor, for example a tenodesis screw, a knotless anchor, a button, an all elongated tensionable structure-anchor, and/or the like described elsewhere herein.

It should be understood that while the anchor 1214 is described herein as being an interference screw, it is contemplated that the anchor 1214 may be any suitable anchor described elsewhere herein, including for example a tenodesis screw, a knotless anchor, a button anchor, an all-suture anchor, or the like.

As shown in FIG. 12C, in some embodiments the tunnel 1210 is also configured to receive at least a portion of the first portion 402. For example, the second end 410 is shown to be manipulated into and/or through the tunnel 1210. In some embodiments, the second end 410 is manipulated into and/or through the tunnel 1210 via manipulation of the tails and/or the lead elongated tensionable structures 1212. As described with reference to FIG. 12B, the lead elongated tensionable structures 1212 are shown to extend through the tunnel 1210 and/or to an end of the tunnel 1210 to a lateral side of the acromion 120. Further, the lead elongated tensionable structures 1212 are shown to be secured to a lateral side of the acromion 120 at an end of the tunnel 1210 via the anchor 1214, as described herein. In this regard, the technique illustrated in FIG. 12C may be implemented in instances where the graft (e.g., the first portion 402, etc.) is relatively longer, for example compared to the technique illustrated in at least FIG. 12B.

As shown in FIG. 12D, in other embodiments the second end 410 may be manipulated into and/or through the tunnel 1210 to an end of the tunnel 1210. In this sense, in some embodiments the second end 410 is manipulated through the tunnel 1210 to a lateral side of the acromion 120. The second end 410 is shown to be secured to a lateral side of the acromion 120 at an end of the tunnel 1210 via the anchor 1214. As described herein, the anchor 1214 may be an interference screw that is driven into the tunnel 1210 to secure the second end 410 relative to the acromion 120. For example, the anchor 1214 may be an interference screw that is driven into the tunnel 1210 to secure the tissue of the second end 410 and/or elongated tensionable structures or filaments attached to the second end 410 (e.g., the lead elongated tensionable structures 1212 described with reference to at least FIGS. 12B-12C, etc.) between threads of the anchor 1214 and one or more sides of the tunnel 1210. In this regard, the technique illustrated in FIG. 12D may be implemented in instances where the graft (e.g., the first portion 402, etc.) is relatively longer, for example compared to the technique illustrated in at least FIGS. 12B-12C.

As described elsewhere herein, it should be understood that while FIGS. 12B-12D illustrate process where the biceps 150 is modified via a subpectoral technique (e.g., as discussed in FIG. 4A), such that the second end 410 has sufficient length to extend towards and/or into tunnel 1210, it is contemplated that any and/or all of the processes illustrated in FIGS. 12B-12D may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.). For example, using the suprapectoral technique as to these illustrated processes of FIGS. 12B-12D may require elongated tensionable structures of sufficient length to extends towards and/or into tunnel 1210.

In some embodiments, and as shown in FIGS. 12E-12F, two tunnels 1210 are created in the acromion 120, for example extending from a medial/superior portion of the acromion 120, toward a lateral/inferior portion of the acromion 120. In other embodiments, the tunnel 1210 is a single tunnel or another suitable number of tunnels, as described herein, which may be otherwise arranged or configured. For example, in some embodiments the tunnel(s) 1210 extend from a superior portion to an inferior portion of the acromion 120, from a superior portion to/out of an anterior side of the acromion 120, from a superior portion to/out of a lateral side of the acromion 120, and/or another suitable configuration. In some embodiments, one tunnel 1210 may exit one side of the acromion 120, and the other tunnel 1210 may exit a different side of the acromion.

As shown in FIGS. 12E-12F, the tunnels 1210 may receive one or more elongated tensionable structures, elongated tensionable structure tails, or material. For example, the tunnels 1210 may be configured to receive elongated tensionable structure tails, which may extend from whipstitching, shown as lead elongated tensionable structures 1212. The tails may be manipulated through the tunnels 1210, and secured to one another on an opposing side of the bone (e.g., a side of the acromion 120, etc.). In this regard, and as shown in FIGS. 12E-12F, it is contemplated that in some embodiments the second end 410 (e.g., the first portion 402, etc.) is secured to the acromion 120, for example without using an anchor. In other embodiments, anchors, which may be the same as or similar to any anchors described herein, and/or may be secured to a bone using any suitable technique or combinations of techniques described herein, may be utilized to secure the elongated tensionable structures tails or graft within a bone or bone structure (e.g., the acromion 120, etc.). In yet other embodiments, and as described elsewhere herein, any and/or all of the processes described herein may be implemented with the biceps 150 being modified via a subpectoral technique (e.g., as shown in FIG. 12E), a suprapectoral technique (e.g., as shown in FIG. 12F), and/or implementations thereof (e.g., including elongated tensionable structures, filaments, and/or materials extending from a cut end to provide a longer overall length of the graft/material, etc.).

In other embodiments, the second end 410 and/or the first portion 402 are secured to a bone (e.g., the acromion 120, the scapula 104, etc.) using another suitable component, device, and/or technique. For example, and as shown in FIGS. 12G-12H, an anchor or retention anchor, shown as anchor 1222, may be coupled (e.g., secured, attached, fixated, etc.) with the first portion 402 (e.g., the second end 410, etc.) and a bone. In exemplary embodiments, the anchor 1222 is a suture anchor (e.g., an all-suture anchor, like an ICONIX® anchor, etc.). According to exemplary embodiments, the anchor 1222 (e.g., suture anchor, etc.) may be utilized in circumstances where a characteristic of the first portion 402 (e.g., a length of the first portion 402, a length of the first portion 402 routed out of the tunnel 504, for example as a result of attachment of the first portion 402 and/or routing of the first portion 402, etc.) limits the amount of the first portion 402 available to complete a process described herein (e.g., limits an amount of the first portion 402 across the acromion 120, etc.). For example, the anchor 1222 may be utilized in circumstances where the first portion 402 is too short to approach an end (e.g., a lateral end, a lateral edge, etc.) of the acromion 120, for example in instances where a suprapectoral technique is used in modifying the bicep (e.g., as discussed with reference to FIG. 4A and/or as shown in FIG. 12H). That said, it should be understood that any and/or all of the processes described herein may be implemented with the biceps 150 being modified via a subpectoral technique (e.g., as shown in FIG. 12G), a suprapectoral technique (e.g., as shown in FIG. 12G), and/or implementations thereof (e.g., including elongated tensionable structures, filaments, and/or materials extending from a cut end to provide a longer overall length of the graft/material, etc.).

As described elsewhere herein, the anchor 1222 may be secured to the bone via any suitable technique. For example, the anchor 1222 may be secured to the bone (e.g., the acromion 120, etc.) using a whipstitching and/or an onlay technique (e.g., as discussed with reference to FIG. 6D), where the elongated tensionable structures (e.g., elongated tensionable structure tails, etc.) may be used to further stabilize the attachment, and/or may be trimmed and removed. In other embodiments, the anchor 1222 may be secured to the bone (e.g., the acromion 120, etc.) using an inlay technique, and/or another suitable technique described herein.

In yet other embodiments, the second end 410 and/or the first portion 402 are secured to a bone (e.g., the clavicle 102, etc.) using another suitable component, device, and/or technique. For example, and as described elsewhere herein, the tunnel 504 may be prepared in the clavicle 102. As shown in at least FIG. 12I, the tunnel 504 extends from an inferior portion of the clavicle 102 to a superior portion of the clavicle 102. According to an exemplary embodiment, the tunnel 504 extends all the way through the clavicle 102. In other embodiments, the tunnel 504 is otherwise arranged and/or configured, for example extending from a lateral portion toward a medial portion, from an anterior portion toward a posterior portion, etc., as described elsewhere herein.

As shown in FIG. 12I, the first portion 402 extends into and/or through the tunnel 504. In exemplary embodiments, the second end 410 is manipulated into and/or through the tunnel 504, and secured relative to the clavicle 102 via an anchor or retention anchor, shown as anchor 1224. In some embodiments, the anchor 1224 is secured to the clavicle 102 by being driven into the tunnel 504, as described elsewhere herein. As will be described herein, the anchor 1224 may be a top hat, a drop crotch button, or a button anchor, for example to facilitate securing the anchor 1224 and the first portion 402 (e.g., the second end 410) within the tunnel 504. In other embodiments, the anchor 1224 and/or the anchor 304 is/are other suitable anchors, for example knotless anchors, all-suture anchors, and/or the like described elsewhere herein. It should be understood that while FIG. 12I illustrates the biceps 150 as being modified via a subpectoral technique (e.g., as discussed in FIG. 4A), it is contemplated that the process illustrated in FIG. 12I may be implemented with the biceps 150 being modified via another suitable technique described herein, including the suprapectoral technique (e.g., as discussed in FIG. 4B) and/or implementations thereof (e.g., including elongated tensionable structures, filaments, and/or materials extending from a cut end to provide a longer overall length of the graft/material, etc.).

It should also be understood that the second end 410 (e.g., the first portion 402, etc.) may be prepared, manipulate, routed, and/or stabilized, for example using any and/or combinations of the techniques described herein (e.g., any and/or all combinations of techniques described with reference to FIGS. 6A-12I). For example, it is contemplated that in one exemplary instance, the first end 152 may be attached to the glenoid rim 126 (e.g., via an inlay technique as shown in FIG. 6C, or an onlay technique as shown in FIG. 6D, etc.), and the second end 410 may be routed around the coracoid process 122 (e.g., as shown in FIG. 8C, etc.) or through the coracoid process 122 (e.g., as shown in FIG. 10A), through the tissue 162 (e.g., through a medial portion of the supraspinatus tissue, for example similar to as illustrated in FIG. 9, etc.) or around the tissue 162, and to/through the tunnel 504 (e.g., with or without the first portion 402 being secured relative to the coracoacromial ligament 160, etc.). The second end 410 may further be manipulated across the clavicle 102 to an anchor point at the scapula 104 (e.g., the acromion 120, etc.), and secured to the anchor point, for example using an anchor (e.g., as shown in FIGS. 12A-D and/or FIGS. 12G-12H) or an anchor-less technique (e.g., as shown in FIGS. 12E-12F). In some instances, the first portion 402 may also be secured relative to the clavicle 102, for example using an anchor (e.g., as shown in FIG. 11).

Further, it should be understood that additional techniques and/or processes may be implemented to further secure and/or stabilize the ligaments, tissues, bones, joints, and/or other features of the shoulder 100. For example, in some embodiments, one or more surrounding tissues may be secured (e.g., stitched, fixated, attached, etc.) to the graft, for example to provide increased stability and/or stabilization. For example, in some embodiments, one or more tissues (e.g., a fascia of the trapezius, a facia of the deltoid, etc.) may be secured (e.g., stitched, fixed, attached, coupled, etc.) to the graft to provide increased stability and/or stabilization (e.g., for the shoulder or joint repair, etc.). The tissues may be secured to the graft, for example using any of the elongated tensionable structures, anchors, or stabilizing techniques described herein. In some embodiments, securing the trapezius and/or the deltoid to the graft may further stabilize the repair and/or further reduce the clavicle 102.

Referring generally to FIGS. 13A-14, illustrations of various devices are shown, according to example embodiments. In exemplary embodiments, the devices of FIGS. 13A-14 may be prosthetic devices, which may be implemented in one or more of the embodiments illustrated in FIGS. 3A-12I. According to some embodiments, the devices of FIGS. 13A-14 are implemented in combination with one or more of the components and/or features described with reference to FIGS. 3A-12I (e.g., elongated tensionable structures, anchors, top hats, buttons, tissues, tendons, ligaments, etc.), for example to facilitate shoulder repair and/or stabilization.

Referring generally to FIGS. 13A-14, a device, shown as a plate 1300, is shown, according to exemplary embodiments. In exemplary embodiments, the plate 1300 is configured to couple one or more bones or bone structures. For example, the plate 1300 may be configured to secure (e.g., via one or more anchors or screws, etc.) one or more bones or bone structures of the clavicle 102 (e.g., a fractured clavicle, etc.) to repair and/or stabilize the clavicle 102 (e.g., repair due to a fracture of the clavicle 102, etc.).

As shown, the plate 1300 includes a body 1302 and a hook 1304. The body 1302 may include a plurality of openings or apertures. The body 1302 may be configured to couple one or more bones or bone structures (e.g., fractured or damaged bones or bone structures of the clavicle 102, etc.), and/or receive one or more anchors or screws (e.g., via the openings or apertures, etc.)to stabilize the bones or bone structures (e.g., repair and/or stabilize the clavicle 102, etc.). The hook 1304 may extend from (e.g., below, interior to, etc.) the body 1302, and/or may be configured to couple one or more bones or bone structures (e.g., of the scapula 104). For example, the hook 1304 may be configured to couple the acromion 120 (e.g., an inferior surface or portion of the acromion 120, etc.) to reduce (e.g., decrease, etc.) a loss of reduction (e.g., distance, vertical loss of reduction, etc.) between the clavicle 102 and the scapula 104.

In some embodiments, the plate 1300 may further be configured to couple one or more additional features of a shoulder or joint (e.g., the shoulder 100, etc.). For example, in certain instances the plate 1300 may be configured to engage a graft (e.g., tendon, ligament, muscle, tissue, etc.) at a surface of the plate 1300 (e.g., a lower surface, a lower groove, a lower channel, etc.). For example, the plate 1300 may include an edge that is elevated, thereby forming a channel, groove, or tunnel at a lower portion or surface of the edge. In exemplary embodiments, the plate 1300 may be positioned and/or configured such that the plate 1300 sits above a tissue or graft (e.g., with the tissue or graft within the lower channel, groove, or tunnel, etc.), thereby allowing the plate 1300 to be removed, without altering the positioning, placement, and/or healing (e.g., growth, scarring, etc.) of the graft (e.g., the biceps 150, etc.).

As shown in FIG. 13A, and as described herein, the plate 1300 may be configured to engage with, interface with, and/or be positioned relative to a portion of the biceps 150 (e.g., the second end 410 of the first portion 402, etc.). As shown in FIG. 13A, a portion of the plate 1300 may be configured to sit above a tissue or graft, such as the first portion 402 of the biceps (shown in FIG. 13A and FIG. 13B). According to various aspects, the plate 1300 may be configured such that the plate 1300 interfaces with a portion of the tissue or graft (e.g., in a groove, channel, etc. at a lower surface of the plate 1300), for example as part of the joint repair and/or stabilization techniques described herein. Having the plate 1300 positioned above the portion of the tissue or graft allows the tissue or graft to be positioned as needed without the plate interfering with the tissue or graft, while also allowing for removal of the plate 1300 without interfering with the tissue or graft. Furthermore, as shown in FIG. 13B, a less wide plate (e.g., the plate 1300, etc.) is envisioned that would allow the graft to be positioned adjacent to the outer edge of the plate 1300, thereby also allowing the plate 1300 to be removed without altering the positioning, placement, and/or healing (e.g., growth, scarring, etc.) of the graft (e.g. the biceps 150, etc.). It should be understood that in some embodiments, the plate 1300 is configured to otherwise engage and/or be positioned relative to a portion of the biceps (e.g., the second end 410 of the first portion 402, etc.).

As shown in FIG. 14, in some embodiments the plate 1300 includes a recess, groove, or channel, shown as channel 1306. According to some embodiments, the channel 1306 is configured to receive a graft or component described herein (e.g., tendon, ligament, elongated tensionable structure, anchor, etc.). For example, the channel 1306 may be configured to receive the second end 410 of the first portion 402 (e.g., of the biceps 150, etc.), thereby allowing the second end 410 to extend from the plate 1300. In exemplary embodiments, the channel 1306 permits the plate 1300 to receive the biceps 150 (e.g., a second end 410 of the first portion 402, etc.) as described herein, while also facilitating removal of the plate 1300, without altering the positioning, placement, and/or healing (e.g., growth, scaring, etc.) of the biceps 150 relative to the bone (e.g., the clavicle 102, etc.). In other embodiments, the plate 1300 includes a plurality of channels 1306 positioned at the body 1302 and/or the hook 1304, which may be configured to receive one or more components or features (e.g., anchors, elongated tensionable structures, tissues, etc.) described elsewhere herein.

In other embodiments, the plate 1300 is or includes additional, fewer, and/or different components. For example, in some embodiments the plate 1300 includes one or more openings, apertures, and/or channels, which may be sized to receive one or more components (e.g., one or more elongated tensionable structures, tissues, grafts, anchors, etc.) described elsewhere herein. In some embodiments, plate 1300 includes one or more surfaces or interfaces (e.g., a protective sheath, rounded or curved edges, etc.), for example to protect components or features received therein (e.g., an elongated tensionable structure, tissue, ligament, etc.) against damage from engaging adjacent bone interfaces (e.g., edges, etc.).

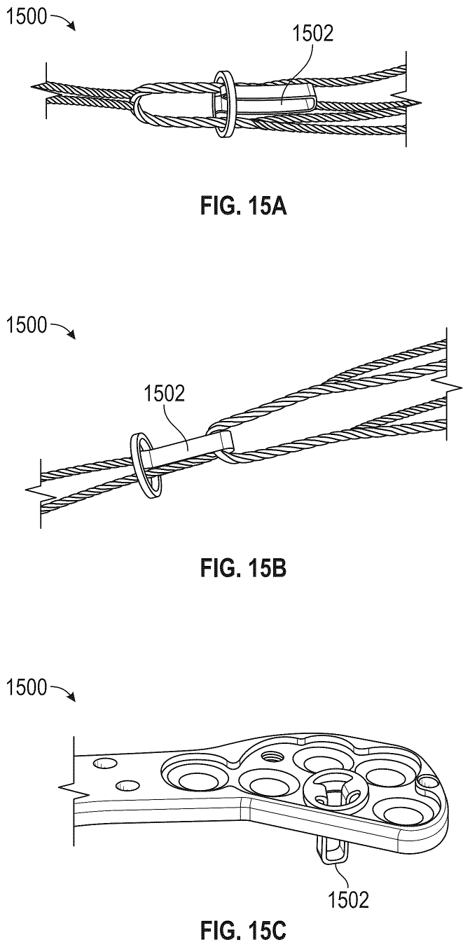

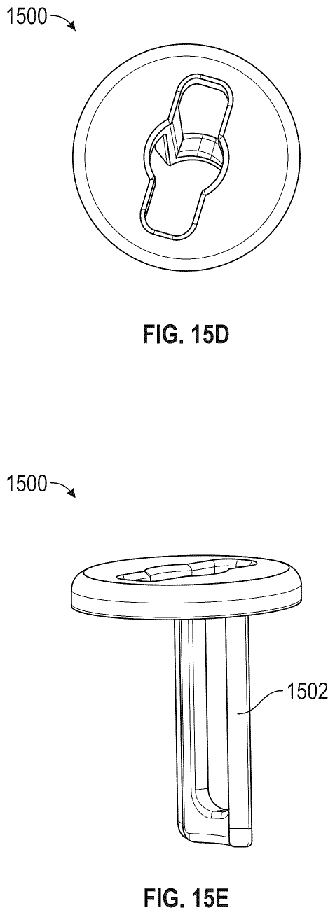

In yet other embodiments, the plate 1300 may be, include, and/or be used in combination with one or more clips, guides, tubes, channels, etc. For example, the plate 1300 may be used in combination with a drop crotch button (e.g., as shown in at least FIGS. 15A-15E as button 1500). The drop crotch button (e.g., button 1500) may include a drop crotch portion, shown as portion 1502, configured to sit in a hole in a plate (e.g., as shown in FIG. 15C) and/or a bone. Further, the drop crotch button may include a drop crotch portion configured to receive an elongated tensionable structure and/or graft (e.g., as shown in FIGS. 15A-15B), for example to allow a knot stack to sit below a bone surface. In some embodiments, the portion 1502 is offset from a center or central axis of the button 1500 (e.g., offset from a central hole or opening in a top of the button 1500, as shown in at least FIGS. 15D-15E).

According to exemplary embodiments, the arrangement and/or configuration of the portion 1502 may allow the button 1500 to receive an elongated tensionable structure and a graft through the same opening or tunnel (e.g., as discussed with regard to at least FIG. 12E). For example, the arrangement and/or configuration of the portion 1502 may allow an elongated tensionable structure (e.g., the elongated tensionable structure 306, etc.) to be received at a side of the button 1500 (e.g., an interior surface or side of the portion 1502, etc.), as well as a graft (e.g., the second end 410, the first portion 402, etc.) at a central or middle portion of the button 1500 (e.g., a central or middle cavity or tunnel, etc.).

It should be understood that the anchors described herein may be or include the button 1500. In this sense, in some embodiments the button 1500 is configured such that a graft extends through the button 1500, or an opening in the button 1500, for example to allow the graft to extend from the button 1500 to be secured relative to a bone, as described herein. In some embodiments, and as described elsewhere herein, the button 1500 may be secured within a tunnel. Further, one or more elongated tensionable structures, tails, and/or stitching may be secured to the button 1500. For example, and as described herein, elongated tensionable structure tails extending from an end of the graft may be secured to the button 1500 to secure the elongated tensionable structure tails and/or graft relative to the button 1500 and/or within the tunnel. In some embodiments, the tunnel is configured to receive another anchor or screw (e.g., an interference screw), for example below the button 1500 within the tunnel. In this sense, in some embodiments the elongated tensionable structure tails may extend through an opening in the anchor or screw situated within the tunnel, and may be attached to the button 1500 to secure the elongated tensionable structure and/or graft relative to the button 1500, the screw, and/or within the tunnel. All such variations and embodiments are contemplated herein.

In some embodiments, the button 1500 may be or include a top hat, for example to receive an elongated tensionable structure and/or tissue, while preventing tunnel enlargement and reducing the effects of knot stacking and/or bone damage. In some embodiments, the plate 1300 is or includes a slotted button, for example to allow elongated tensionable structures, tissues, and/or filaments to pass through the plate 1300 and then a button to be attached. In yet other embodiments, the plate 1300 is otherwise arranged or configured. All such embodiments are contemplated herein.

According to exemplary embodiments, the systems and methods described herein provide a joint repair and/or stabilization technique, which may provide improved clinical outcomes, more repeatable and/or successful repairs, reduced costs, and/or decreased time in surgery. More specifically, and as described with reference to FIGS. 3A-15, the process described herein utilizes a tissue autograft (e.g., a long head of the biceps tendon, etc.), which is routed from an anchor point at a first bone (e.g., a point at a tubercle of the glenoid cavity of the scapula, etc.), to and/or through a second bone (e.g., the clavicle, etc.), across the second bone (e.g., the clavicle, etc.), and back to the first bone to a second anchor point (e.g., an acromion of the scapula, etc.). Advantageously, this routing provides for tensioning of the joint, while facilitating a simplified anatomical reduction process that reduces the risks associated with current techniques (e.g., eliminates knot stacking by eliminating elongated tensionable structure-anchor interfaces, potential failures associated with synthetic elongated tensionable structure tensioning techniques, etc.) and/or leads to improved healing (e.g., via the use of an autograft, etc.). Further, aspects or embodiments advantageously utilize an existing anatomical anchor point (e.g., existing anchoring of the long head of the biceps tendon at a tubercle of the glenoid cavity of the scapula, etc.), which further can lead to improved healing and/or improved stability, compared to traditional techniques.

It should be understood that while the systems and methods are described herein as having certain components, features, and/or structures, it is contemplated that in other embodiments the systems and methods can be used with additional, fewer, and/or different components, features, and/or structures. For example, in some embodiments, in addition to routing an autograft tissue (e.g., a portion of the biceps, etc.), the systems and methods described herein could be implemented by routing additional augmentation material (e.g., a synthetic augment material, an allograft tissue, an autograft tissue combined with a synthetic and/or allograft tissue, etc.), as described herein. For example, any and/or all of the techniques described herein may be implemented using an autograft (e.g., a hamstring autograft, etc.), an allograft (e.g., a hamstring allograft, etc.), or another suitable tissue.

In other embodiments, rather than routing an autograft tissue (e.g., a portion of the biceps, etc.), the systems and methods described herein may be implemented by routing different augmentation material (e.g., a synthetic augment material, an allograft tissue, etc.) in place of the autograft tissue. For example, an allograft tissue (e.g., allograft tendon, etc.) and/or synthetic tissue (e.g., polyester braids, biocompatible tape, tape having tissue-like properties, etc.) may be anchored at a first bone (e.g., at a glenoid cavity of the scapula, at a portion of the scapula, etc.), routed to and/or through a second bone (e.g., the clavicle, etc.), and secured back to the first bone (e.g., at an acromion of the scapula, etc.), as described herein. One such material may include the aforementioned FlexBand® material, which can replace or further reinforce or augment the soft tissue autograft/allograft material.

In yet other embodiments, the systems and methods described herein may be implemented with one or more hybrid and/or combination material (e.g., a synthetic-augment hybrid, a synthetic-allograft material hybrid, a biocomposite material, etc.), which may be used in combination with the allograft tissue described herein and/or independently. All such embodiments are contemplated herein, which may be implemented in any and/or all of the steps and/or configurations shown in the exemplary illustrative diagrams of FIGS. 3A-15.

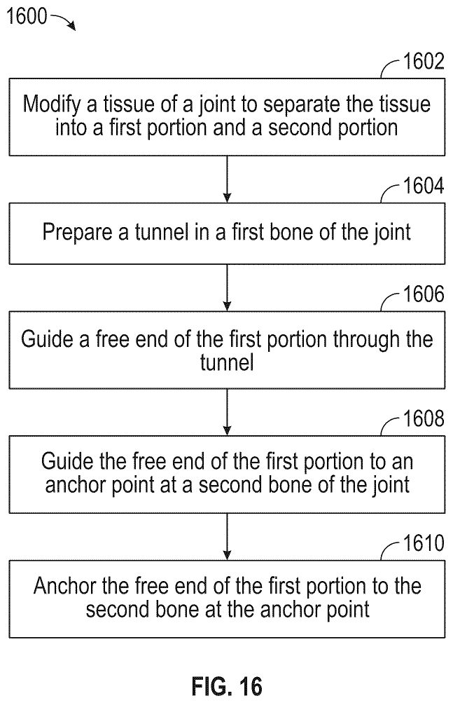

Referring now to FIG. 16, a process for repairing, stabilizing, and/or modifying a joint, shown as a process 1600, is shown, according to an exemplary embodiment. The process 1600 may be implemented for repairing, stabilizing, and/or modifying a joint, for example the shoulder 100 of FIG. 1 and/or the shoulder 204 of FIG. 2. In exemplary embodiments, the process 1600 can be implemented to provide any and/or all of the steps and/or configurations shown in the exemplary illustrative diagrams of FIGS. 3A-15E.

Process 1600 is shown to include modifying a tissue of a joint to separate the tissue into a first portion and a second portion (1602), according to an exemplary embodiment. In exemplary embodiments, the process 1600 includes modifying (e.g., cutting, slicing, severing, dividing, etc.) a biceps tissue into a first portion (e.g., a proximal portion, etc.) and a second portion (e.g., a distal portion, etc.). For example, the process 1600 may include performing a tenotomy on long head of the biceps tendon, for example to divide (e.g., cut, sever, etc.) the long head of the biceps tendon into a first portion and a second portion.

As described herein, the first portion may include a first end coupled with (e.g., anchored to, etc.) a first bone (e.g., the scapula, a tubercle of the glenoid cavity, etc.) and a second end that is free. As described herein, the first end (e.g., of the first portion, etc.) may be anchored with the first bone at a native location (e.g., via a native attachment, reattachment to a native location, etc.) and/or another suitable location at the first bone. Further, the second portion may include a first end, which following the modification of the tissue is a free end, and a second end that is coupled with (e.g., anchored to, etc.) another bone. As described herein, the second portion (e.g., a first end of the second portion, etc.) may be coupled with a bone (e.g., the humerus, etc.), for example by interlacing a tissue junction of the second portion with elongated tensionable structures and coupling (e.g., anchoring, etc.) the elongated tensionable structures to the bone (e.g., humerus, etc.) via an anchor.

As also described herein, the process 1600 may include modifying the biceps tendon (e.g., performing a tenotomy or tenodesis on the long head of the biceps tendon, etc.) via any suitable surgical approach. For example, the process 1600 (e.g., 1602) may include modifying the biceps tendon via a subpectoral tenodesis approach, for example to ensure a predetermined characteristic (e.g., length, size, etc.) of the first portion (e.g., via a subpectoral tenodesis approach close to the musculotendinous junction, for example to preserve a longer length of the first portion, etc.). In other embodiments, the process 1600 (e.g., 1602) includes modifying the biceps tendon via a suprapectoral tenodesis approach, as described with reference to FIG. 4B, for example to ensure a predetermined characteristic (e.g., length, size, etc.) of the first portion (e.g., via a suprapectoral tenodesis approach, for example resulting in a shorter length of the first portion, etc.).

In some embodiments, the process 1600 includes performing one or more diagnostic techniques. For example, a diagnostic process may be implemented on a joint to assess one or more joint conditions and/or characteristics (e.g., pathogens, concomitant glenohumeral pathologies, inflammation of a tissue or tendon, etc.). In some embodiments, the joint conditions and/or characteristics are used in modifying the tissue (e.g., in 1602, discussed herein). In this regard, the process 1600 may include performing one or more diagnostic techniques prior to modifying the tissue (e.g., in 1602), for example to provide information and/or guidance to perform the modification of the tissue.

In some embodiments, the process 1600 includes performing a tensioning technique. In some embodiments, the process 1600 includes performing a tensioning technique to reduce a loss of reduction (e.g., distance, vertical loss of reduction, etc.) between the clavicle and the scapula (e.g., coracoid process, a coracoclavicular distance, etc.). For example, the process 1600 may include performing a tensioning technique using one or more anchors at one or more bones (e.g., a first anchor at the clavicle, a second anchor at the scapula, etc.), and/or an elongated tensionable structure extending between the one or more anchors and/or the one or more bones. In some embodiments, the process 1600 may include coupling a first anchor to the clavicle, coupling a second anchor to the scapula (e.g., a coracoid process of the scapula, etc.), coupling an elongated tensionable structure to the first anchor and the second anchor, and applying a force to the elongated tensionable structure (e.g., tensioning the elongated tensionable structure, etc.), for example to reduce a distance between the clavicle and the scapula (e.g., a coracoclavicular distance, etc.). It should be understood that in some embodiments, the elongated tensionable structure is coupled to one or more anchors (e.g., the first anchor and/or the second anchor, etc.), for example via another entity (e.g., a manufacturer, a third-party, etc.) that supplies and/or provides an elongated tensionable structure/anchor combination.

As described herein, in some embodiments the process 1600 includes performing a tensioning technique that implements additional, fewer, and/or different components. For example, the tensioning technique may involve coupling an anchor to a first bone (e.g., the clavicle, the scapula, etc.), coupling an elongated tensionable structure to the anchor, extending the elongated tensionable structure to, around, and/or through a second bone (e.g., the scapula, the clavicle, etc.), and tensioning the elongated tensionable structure to decrease a distance between the first bone and the second bone. In some embodiments, the tensioning technique involves coupling an elongated tensionable structure to a first bone (e.g., by looping the elongated tensionable structure to, around, or through the first bone, etc.), extending the elongated tensionable structure to, around, and/or through a second bone, and tensioning the elongated tensionable structure to decrease a distance between the first bone and the second bone. In some embodiments, the process 1600 may include performing a tensioning technique prior to modifying the tissue (e.g., in 1602), for example to reduce a force exerted on (e.g., tension applied, etc.) the tissue being modified.

Process 1600 is shown to include preparing a tunnel in first bone of the joint (1604), according to an exemplary embodiment. In exemplary embodiments, the process 1600 includes preparing (e.g., cutting, drilling, resecting, etc.) a tunnel in a clavicle of the joint. The tunnel may be prepared via a guidewire, a drill, and/or another suitable surgical device described herein.

As discussed herein, in some embodiments the process 1600 includes preparing a plurality of tunnels or holes in a first bone of the joint. For example, the process 1600 may include preparing a first hole in the clavicle (e.g., to receive a first anchor, etc.), for example as part of the tensioning technique described herein. Further, the process 1600 may include preparing a second hole or tunnel (e.g., in 1604), for example to receive a portion of the modified tissue (e.g., the first portion of the modified tissue, etc.), as will be discussed herein. In such embodiments, the two holes or tunnels may be separated and/or positioned relative to one another in a predetermined configuration (e.g., a 5 mm, 7 mm, 10 mm, etc. distance between), for example to ensure the structural integrity and/or stability of the bone (e.g., the clavicle, etc.).

In other embodiments, the process 1600 includes preparing a single tunnel or hole in the first bone of the joint. For example, the process 1600 may include preparing a tunnel in the clavicle, for example to receive a first anchor and/or an elongated tensionable structure (e.g., as part of a tensioning technique, etc.) and a portion of the modified tissue (e.g., the first portion of the modified tissue, etc.), as will be discussed herein. In this regard, in such embodiments, the process 1600 may include preparing the tunnel with a component or device (e.g., a clip, a button, a tube, K-wires, a plate, a frame, etc.), for example to facilitate receiving the anchor, the elongated tensionable structure, and/or the modified tissue.

As also discussed herein, in some embodiments the process 1600 includes preparing the tunnel with one or more components or devices (e.g., a protective sheath, a clip, a frame, a plate, a tube, etc.), for example to protect against damage from engaging adjacent bone interfaces (e.g., edges, etc.). In other embodiments, the process 1600 includes preparing one or more additional features of the joint. For example, the process 1600 may include preparing an end of the modified tissue (e.g., measuring a diameter of an end of the modified tissue, trimming an end of the modified tissue, interlacing an end of the modified tissue with one or more elongated tensionable structures, etc.).

Process 1600 is shown to include guiding the first portion through the tunnel (1606), according to an exemplary embodiment. In exemplary embodiments, the process 1600 includes guiding a free end of the first portion through the tunnel. In some embodiments, the free end of the first portion is guided through the tunnel using one or more surgical devices and/or tools (e.g., a camera, a grasper, etc.).

In some embodiments, the process 1600 includes imparting a force on the first portion. For example, the process 1600 may include guiding the first portion of the modified tissue through the tunnel, and/or imparting a tensioning force on the tissue (e.g., tensioning the modified tissue within the tunnel, etc.). In some embodiments, the process 1600 includes anchoring the first portion to a bone. For example, the process 1600 may include anchoring the first portion of the modified tissue within the tunnel (e.g., via an anchor, etc.). In other embodiments, the first portion of the modified tissue is anchored within the tunnel, for example while the tissue is in tension. In still a further alternative, the first portion may be anchored (e.g., secured, etc.) adjacent to or at a tunnel, bore hole or the like, by an elongated tensionable structure anchor and an elongated tensionable structure, for example, such that the anchor may be positioned in the bone but the modified tissue remains outside of the bone. In this regard, the process 1600 may include anchoring the first portion of the modified tissue within the tunnel and/or relative to the first bone (e.g., the clavicle, etc.) in one or more predetermined conditions (e.g., a tensioned condition etc.).

Process 1600 is shown to include guiding the first portion to an anchor point at a second bone of the joint (1608), according to an exemplary embodiment. In exemplary embodiments, process 1600 includes guiding a free end of the first portion to an anchor point at a lateral portion of the scapula (e.g., the acromion, etc.). For example, the process 1600 (e.g., in 1608) may include guiding a free end of the first portion across the clavicle (e.g., over the clavicle, toward a lateral portion, toward a posterior portion, etc.), and/or to an anchor point at a lateral portion of the scapula (e.g., the acromion). In some embodiments, the free end of the first portion is guided to the anchor using one or more surgical devices and/or tools (e.g., a camera, a grasper, etc.).

In some embodiments, the process 1600 includes guiding the first portion relative to a plate, implant, or other device. For example, in instances where a plate, implant, and/or device is implanted in the first bone (e.g., the clavicle, etc.) and/or the second bone (e.g., the scapula, etc.), the first portion may be guided to and/or positioned at an opening or gap in the plate or implant. In this regard, the first portion of the modified tissue may be positioned relative to an opening or gap in a plate or implant, for example to facilitate removal of the plate or implant, without altering the positioning, placement, and/or growth characteristics of the first portion (e.g., the modified tissue, etc.).

Process 1600 is shown to include anchoring the free end of the first portion to the second bone at the anchor point (1610), according to an exemplary embodiment. In exemplary embodiments, with the free end of the first portion positioned at an anchor point, an anchor may be used to couple the first portion (e.g., the modified tissue) to the second bone (e.g., the scapula, etc.). In exemplary embodiments, the anchor point is a lateral portion of the scapula, for example at the acromion. In some embodiments, the first portion of the modified tissue is anchored to the second bone via a knotless anchor. In other embodiments, the first portion of the modified tissue is anchored (e.g., coupled, secured, attached, etc.) via another suitable anchor described herein (e.g., a staple, a soft tissue stable, an elongated tensionable structure anchor, an all-elongated tensionable structure anchor, for example an ICONIX® anchor, and/or another suitable anchor, etc.). In some embodiments, the first portion is tensioned (e.g., as described herein) prior to anchoring the end of the first portion to the bone.

As described elsewhere herein, the process 1600 may be implemented to provide a joint repair and/or stabilization technique, which may provide improved clinical outcomes, more repeatable and/or successful repairs, reduced costs, and/or decreased time in surgery. More specifically, the process 1600 may utilize a tissue autograft (e.g., a long head of the biceps tendon, etc.) which is routed from an anchor point at a first bone (e.g., a point at a tubercle of the glenoid cavity of the scapula, etc.), to and/or through a second bone (e.g., a tunnel in the clavicle, etc.), across the second bone (e.g., the clavicle, etc.), and back to the first bone to a second anchor point (e.g., an acromion of the scapula, etc.). Advantageously, this routing provides for tensioning at the joint, while facilitating a simplified anatomical reduction process that reduces the risks associated with current techniques (e.g., knot stacking by eliminating elongated tensionable structure-anchor interfaces, potential failures associated with synthetic elongated tensionable structure tensioning techniques, etc.) and/or leads to improved healing (e.g., via the use of an autograft, etc.). Further, this advantageously utilizes an existing anatomical anchor point (e.g., existing anchoring of the long head of the biceps tendon at a tubercle of the glenoid cavity of the scapula, etc.), which further can lead to improved healing and/or improved stability, compared to traditional technique.

As used herein, the term “medial,” when used in connection with a component or a patient's anatomy, refers to a side or region facing or nearer the center of the patient. The term “lateral,” when used in connection with a component or a patient's anatomy, refers to a side or region facing or nearer the patient's side, i.e., away from the center of the patient. As used herein, the term “superior,” when used in connection with a component of a patient's anatomy, refers to a side or region facing or near a top or upper region of the patient. The term “inferior,” when used in connection with a component of a patient's anatomy, refers to a side or region facing or near a lower or bottom region of the patient.

As utilized herein with respect to numerical ranges, the terms “approximately,” “about,” “substantially,” and similar terms generally mean +/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. For example, the stages illustrated and described in the exemplary embodiment described with reference to at least FIGS. 3A-15 may be incorporated in the process of the exemplary embodiment described with reference to at least FIG. 16. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A method for stabilizing a joint having a first bone and a second bone, the method comprising:
modifying a tissue of the joint to separate the tissue into a first portion and a second portion, the first portion of the modified tissue having a first end anchored to the second bone of the joint and a second end that is freely movable;
preparing a tunnel in the first bone of the joint;
guiding the second end of the first portion of the modified tissue through the tunnel;
guiding the second end of the first portion of the modified tissue to an anchor point at the second bone of the joint; and
anchoring the second end of the first portion of the modified tissue to the second bone at the anchor point.

2. The method of claim 1, wherein the joint is an acromioclavicular joint, and the first bone is a clavicle and the second bone is a scapula.

3. The method of claim 1, further comprising:
anchoring a first end of the second portion of the modified tissue to a third bone of the joint, wherein the third bone of the joint is a humerus.

4. The method of claim 1, further comprising:
performing a tensioning operation to draw the first bone and the second bone toward one another.

5. The method of claim 4, wherein the tensioning operation further comprises:
coupling a first anchor to the first bone and a second anchor to the second bone;
coupling the first anchor and the second anchor via an elongated tensionable structure; and
tensioning the elongated tensionable structure to decrease a distance between the first anchor and the second anchor to draw the first bone and the second bone toward one another.

6. The method of claim 4, wherein the tensioning operation further comprises:
coupling an anchor to the first bone or the second bone;
coupling an elongated tensionable structure to the anchor and looping the elongated tensionable structure around a portion of the other of the first bone or the second bone without the anchor; and
tensioning the elongated tensionable structure to decrease a length of the elongated tensionable structure between the first bone and the second bone to draw the first bone and the second bone toward one another.

7. The method of claim 4, wherein the tensioning operation further comprises:
coupling an elongated tensionable structure to a first bone and the second bone, wherein coupling the elongated tensionable structure to the first bone and the second bone includes looping the elongated tensionable structure around a portion of at least the first bone or the second bone; and
tensioning the elongated tensionable structure to decrease a length of the elongated tensionable structure between the first bone and the second bone to draw the first bone and the second bone toward one another.

8. The method of claim 4, wherein the tensioning operation further comprises:
coupling an anchor to the first bone within the tunnel;
coupling an elongated tensionable structure to the anchor and a portion of the second bone; and
tensioning the elongated tensionable structure to decrease a length of the elongated tensionable structure between the first bone and the second bone to draw the first bone and the second bone toward one another.

9. The method of claim 1, wherein preparing the tunnel includes preparing the tunnel with a protective sheath at an internal surface of the tunnel.

10. The method of claim 1, further comprising:
applying a force to the second end of the first portion of the modified tissue to impart a tension force on the first portion of the modified tissue within the tunnel; and
anchoring the first portion of the modified tissue within the tunnel via a stabilization anchor to stabilize the first portion of the modified tissue relative to the tunnel and the first bone.

11. The method of claim 1, wherein the second end of the first portion is anchored to the anchor point via a knotless anchor configured to couple at least one elongated tensionable structure interlaced with a tissue of the second end of the first portion of the modified tissue.

12. A method for stabilizing a joint having a first bone, a second bone, and an augment material having a first end anchored to the second bone, the method comprising:
preparing the joint by providing the augment material to allow a second end of the augment material, opposite the first end, to be freely movable;
preparing a tunnel in the first bone of the joint;
guiding the second end of the augment material through the tunnel;
guiding the second end of the augment material to an anchor point at the second bone of the joint; and
anchoring the second end of the augment material to the second bone at the anchor point.

13. The method of claim 12, wherein the joint is an acromioclavicular joint, the first bone is a clavicle, and the second bone is a scapula, and wherein the augment material is a synthetic material formed of polyester braids.

14. The method of claim 12, wherein the joint is an acromioclavicular joint, the first bone is a clavicle, and the second bone is a scapula, and wherein the augment material is an allograft tissue.

15. The method of claim 12, wherein the joint is an acromioclavicular joint, the first bone is a clavicle, and the second bone is a scapula, and wherein the augment material an autograft tissue.

16. The method of claim 15, wherein the autograft tissue is a long head of a biceps tendon.

17. The method of claim 15, further comprising:
performing a tensioning operation to draw the first bone and the second bone toward one another.

18. The method of claim 17, wherein the tensioning operation comprises applying a force to an elongated tensionable structure coupled to the first bone and the second bone.

19. The method of claim 15, further comprising:
anchoring the augment material to the first bone via an anchor coupled to the augment material and received within the tunnel.

20. A method for stabilizing a joint having a first bone and a second bone, the method comprising:
severing a tissue of the joint to separate the tissue into a first portion and a second portion, the first portion having a first end anchored to the second bone of the joint and a second end that is freely movable;
performing a tensioning operation by applying a force to an elongated tensionable structure coupled to the first bone and the second bone to draw the first bone and the second bone toward one another;
preparing a tunnel in the first bone of the joint;
guiding the second end of the first portion of the tissue through the tunnel;
anchoring the first portion of the tissue to the first bone via an anchor secured to the first portion of the tissue and received within the tunnel;
guiding the second end of the first portion of the tissue to an anchor point at the second bone of the joint; and
anchoring the second end of the first portion of the tissue to the second bone at the anchor point.
